(19) United States
(12) Patent Application Publication
(10) Pub. No.: US 2014/0336503 A1
(43) Pub. Date: Nov. 13, 2014

(54) RADIOLABELED BIOMARKERS FOR OSTEOCLAST ACTIVATION AND RELATED METHODS THEREOF

(71) Applicant: The Regents of the University of Michigan, Ann Arbor, MI (US)

(72) Inventors: Michael R. Kilbourn, Ann Arbor, MI (US); Kenneth Kozloff, Ann Arbor, MI (US); Melissa E. Rodnick, Novi, MI (US)

(73) Assignee: The Regents of the University of Michigan, Ann Arbor, MI (US)

(21) Appl. No.: 14/266,458

(22) Filed: Apr. 30, 2014

Related U.S. Application Data
(60) Provisional application No. 61/821,834, filed on May 10, 2013.

(51) Int. Cl. A61K 51/04; A61B 6/00; A61B 6/03

(52) U.S. Cl. CPC A61K 51/0459; A61B 6/037; A61B 6/481
USPC 600/425; 544/329; 544/295; 544/328; 544/280; 600/431

(57) ABSTRACT

The present invention provides biomarkers that find use as imaging agents within nuclear medicine applications (e.g., PET imaging and SPECT imaging). In particular, the present invention provides methods for identifying and characterizing osteoclast activation within subjects with radiolabeled cathepsin K inhibitors.

### CROSS-REFERENCE TO RELATED APPLICATIONS

This application claims priority to pending U.S. Provisional Patent Application No. 61/821,834, filed May 10, 2013, the contents of which are incorporated by reference in its entirety.

### STATEMENT REGARDING FEDERALLY SPONSORED RESEARCH OR DEVELOPMENT

This invention was made with government support under AR061594 awarded by the National Institutes of Health. The government has certain rights in the invention.

### FIELD OF THE INVENTION

The present invention provides biomarkers that find use as imaging agents within nuclear medicine applications (e.g., PET imaging and SPECT imaging). In particular, the present invention provides methods for identifying and characterizing osteoclast activation within subjects with radiolabeled cathepsin K inhibitors.

### BACKGROUND

Osteoporosis affects 55% of Americans over the age of 50 and is responsible for millions of musculoskeletal fractures annually. Incidents of osteoporosis are increasing. For example, the number of cases of osteoporosis in the United States in 2010 was approximately 12 million while this number is expected to increase to 14 million by 2020.

Accordingly, improved methods for detecting and characterizing musculoskeletal disorders (e.g., osteoporosis) or potential for the onset for such disorders, are needed.

### SUMMARY

Cathepsin K is a cysteine peptidase primarily located in osteoclasts, cells involved in normal growth and remodeling of bone but that are also responsible for bone loss in osteolytic diseases such as osteoporosis. In vivo imaging of cathepsin K may provide a method to assess changes in osteoclast numbers in such disease states. To that end, experiments conducted during the course of developing embodiments developed high-affinity and selective cathepsin K inhibitors radiolabeled with carbon-11 (see, e.g., FIG. 1). It was shown that in vivo microPET imaging studies demonstrated uptake and prolonged retention of radioactivity in actively growing or remodeling bone regions (e.g., distal ulnar, carpal, distal and proximal humeral, distal femur, proximal tibia, tail vertebrae). It was further shown that uptake into bone could be blocked by pre- or co-injection of unlabeled ligand, supporting a specific and saturable binding mechanism for radiotracer localization. These proof-of-concept studies indicate that radiolabeled cathepsin K inhibitors have potential as in vivo imaging radiotracers for assessing changes of osteoclast numbers in osteolytic diseases.

Accordingly, the present invention provides molecular imaging radiotracers for osteoporosis and other musculoskeletal diseases (e.g., osteoarthritis, rheumatoid arthritis, osteopetrosis) involving potential bone loss. In some embodiments, the present invention provides a series of radiolabeled inhibitors based on cyanopyrimidine and pyrrolopyrimidine scaffolds, using isotopic (carbon-11) substitution or synthesis of fluorinated analogs (for fluorine-18 labeling). Such inhibitors have high affinity for human cathepsin K (<10 nanomolar), excellent selectivity over cathepsins L and S (100-1000 fold selective), and all are readily labeled using carbon-11, fluorine-18 or iodine-123.

For example, in certain embodiments, the present invention provides compositions comprising a radiolabeled cathepsin K inhibitor. Examples of such radiolabeled cathepsin K inhibitors include, but are not limited to,

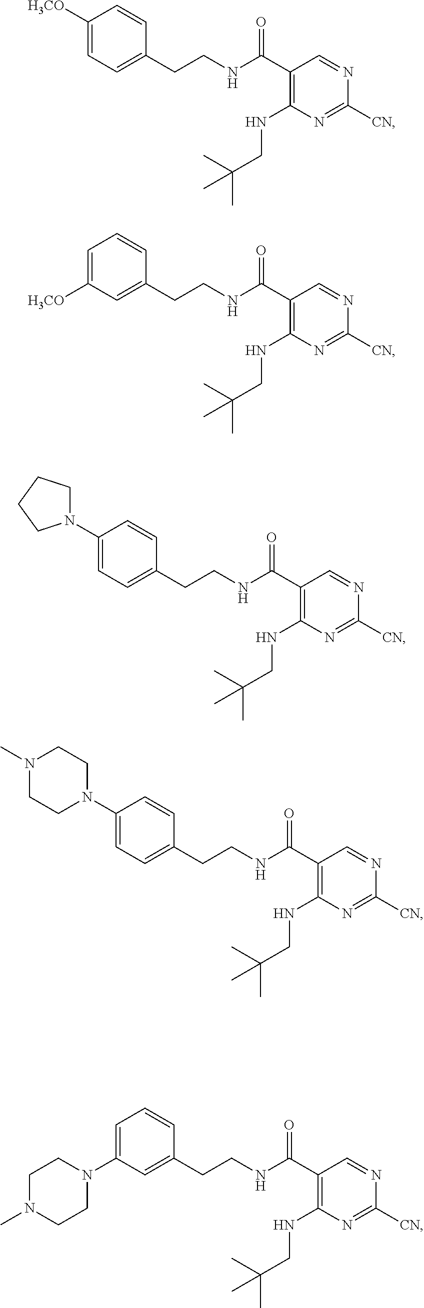 [SMILES: COc1ccc(CCNC(=O)c2cnc(C#N)nc2NCC(C)(C)C)cc1 ; COc1cccc(CCNC(=O)c2cnc(C#N)nc2NCC(C)(C)C)c1 ; CC(C)(C)CNc1nc(C#N)ncc1C(=O)NCCc1ccc(N2CCCC2)cc1 ; CN1CCN(c2ccc(CCNC(=O)c3cnc(C#N)nc3NCC(C)(C)C)cc2)CC1 ; CN1CCN(c2cccc(CCNC(=O)c3cnc(C#N)nc3NCC(C)(C)C)c2)CC1]

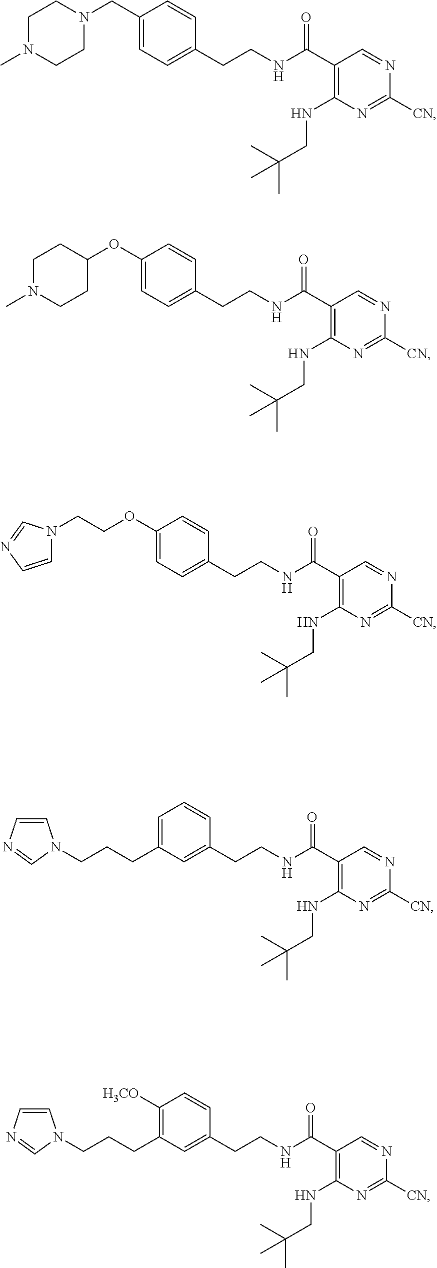 [SMILES: CN1CCN(Cc2ccc(CCNC(=O)c3cnc(C#N)nc3NCC(C)(C)C)cc2)CC1 ; CN1CCC(Oc2ccc(CCNC(=O)c3cnc(C#N)nc3NCC(C)(C)C)cc2)CC1 ; CC(C)(C)CNc1nc(C#N)ncc1C(=O)NCCc1ccc(OCCn2ccnc2)cc1 ; CC(C)(C)CNc1nc(C#N)ncc1C(=O)NCCc1cccc(CCCn2ccnc2)c1 ; COc1ccc(CCNC(=O)c2cnc(C#N)nc2NCC(C)(C)C)cc1CCCn1ccnc1]

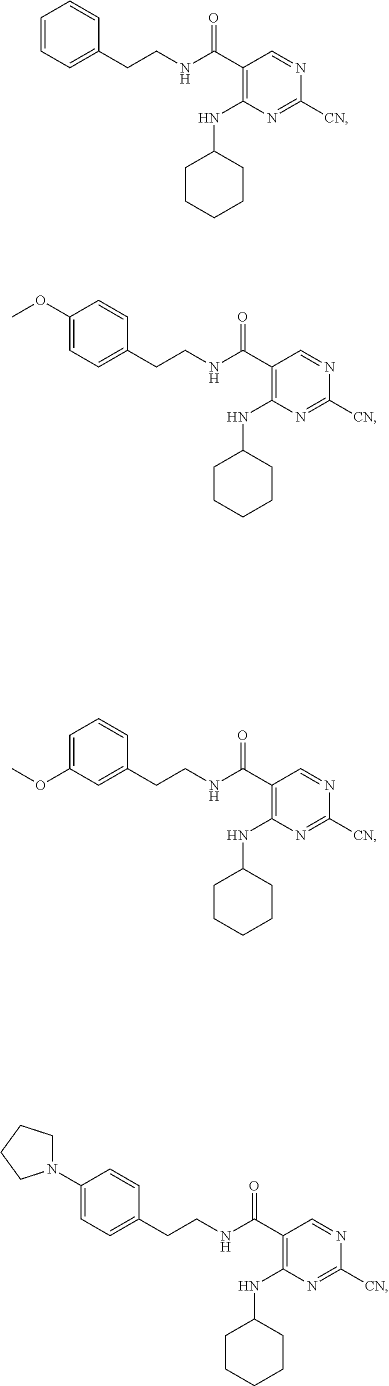 [SMILES: N#Cc1ncc(C(=O)NCCc2ccccc2)c(NC2CCCCC2)n1 ; COc1ccc(CCNC(=O)c2cnc(C#N)nc2NC2CCCCC2)cc1 ; COc1cccc(CCNC(=O)c2cnc(C#N)nc2NC2CCCCC2)c1 ; N#Cc1ncc(C(=O)NCCc2ccc(N3CCCC3)cc2)c(NC2CCCCC2)n1]

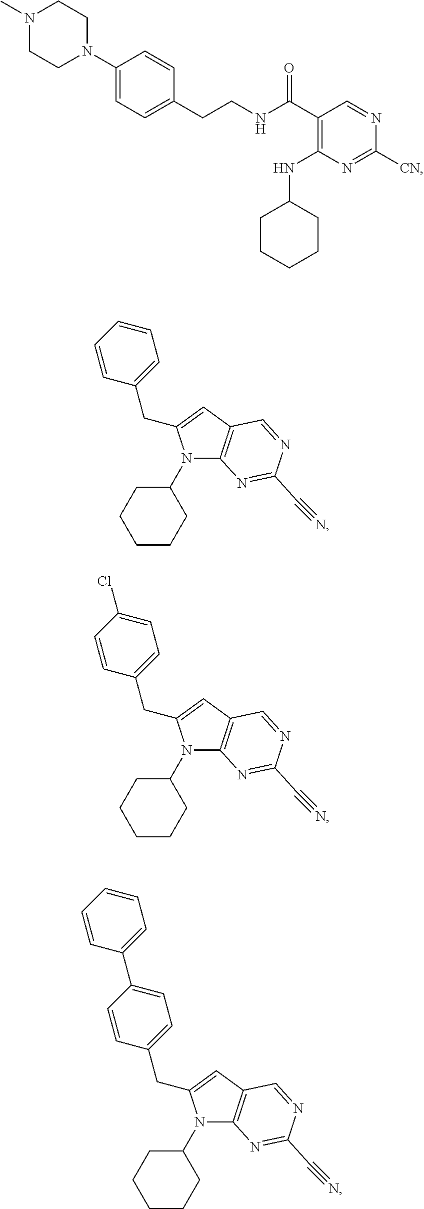

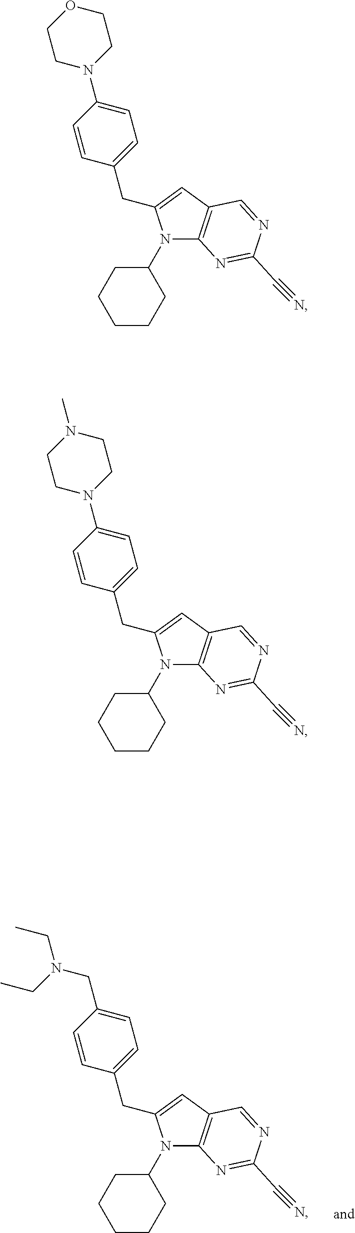 [SMILES: N#Cc1ncc2cc(Cc3ccc(N4CCOCC4)cc3)n(C3CCCCC3)c2n1 ; CN1CCN(c2ccc(Cc3cc4cnc(C#N)nc4n3C3CCCCC3)cc2)CC1 ; CCN(CC)Cc1ccc(Cc2cc3cnc(C#N)nc3n2C2CCCCC2)cc1]

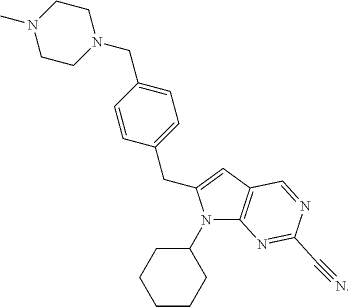 [SMILES: CN1CCN(Cc2ccc(Cc3cc4cnc(C#N)nc4n3C3CCCCC3)cc2)CC1]

Such compounds are not limited to a particular manner of radiolabeling. In some embodiments the radiolabeling is with ¹¹_{C,} ¹⁸F, or ¹²³I.

In some embodiments, the radiolabelling is as shown in FIG. 1.

In some embodiments, the compounds are radiolabelled in the following manner:

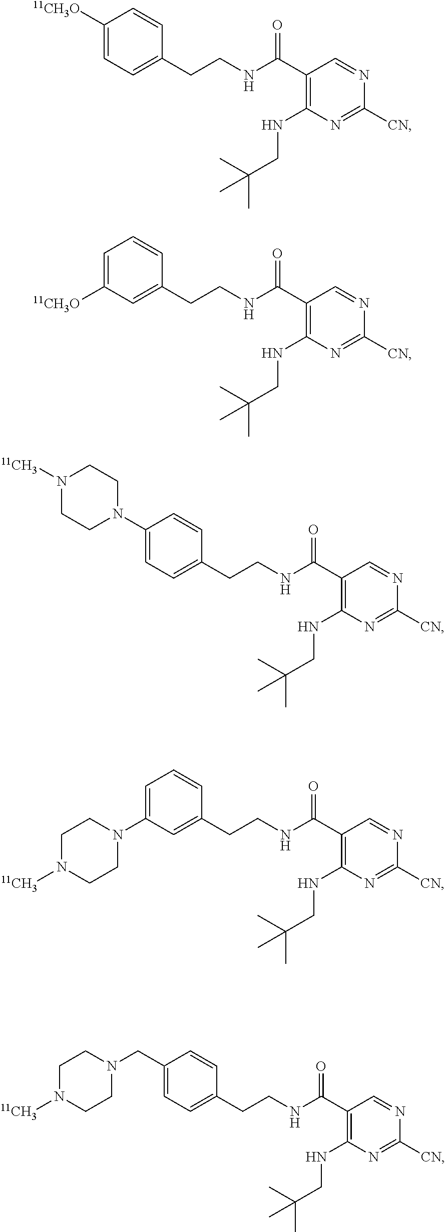

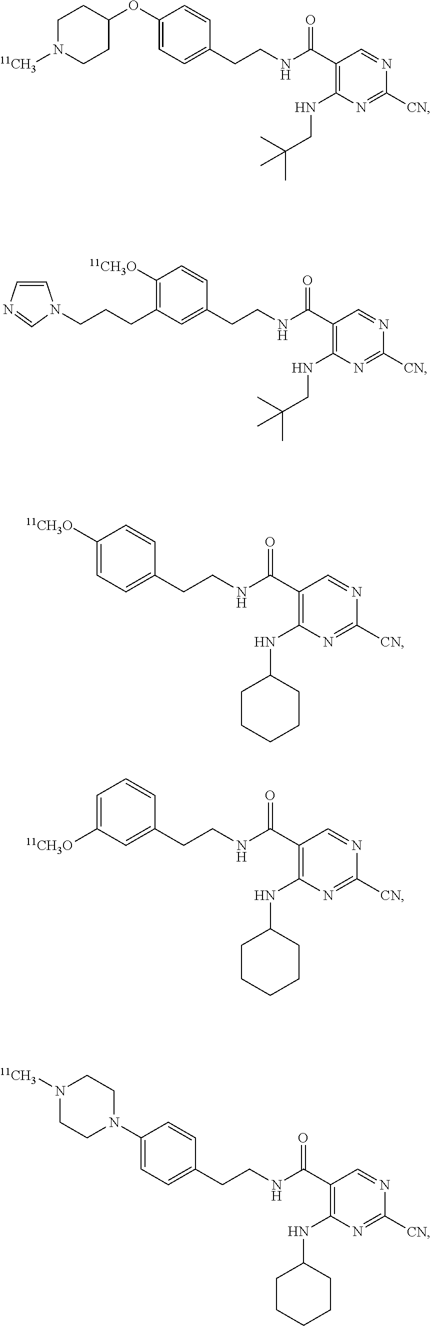

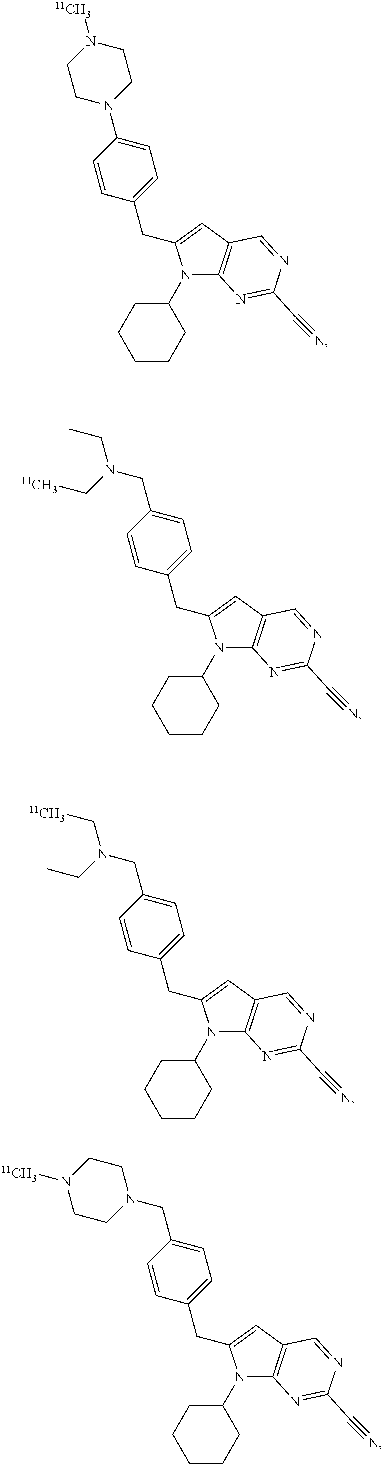 [SMILES: [11CH3]N1CCN(c2ccc(Cc3cc4cnc(C#N)nc4n3C3CCCCC3)cc2)CC1 ; CCN(C[11CH3])Cc1ccc(Cc2cc3cnc(C#N)nc3n2C2CCCCC2)cc1 ; CCN(C[11CH3])Cc1ccc(Cc2cc3cnc(C#N)nc3n2C2CCCCC2)cc1 ; [11CH3]N1CCN(Cc2ccc(Cc3cc4cnc(C#N)nc4n3C3CCCCC3)cc2)CC1]

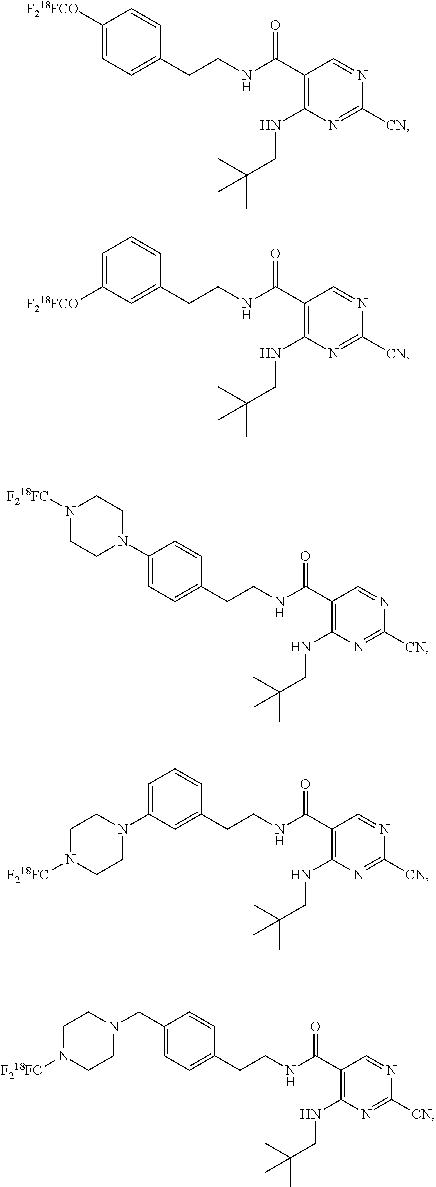

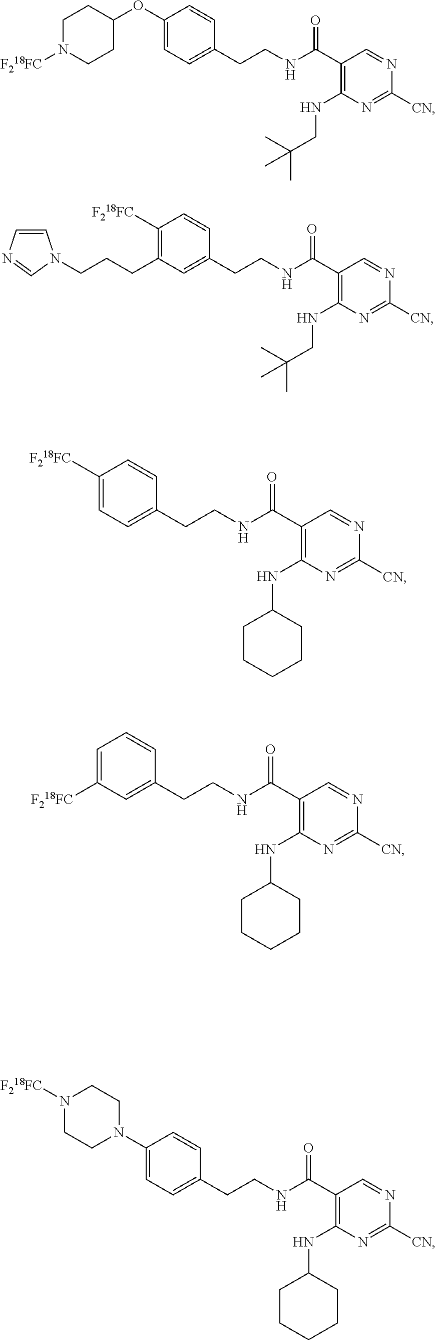

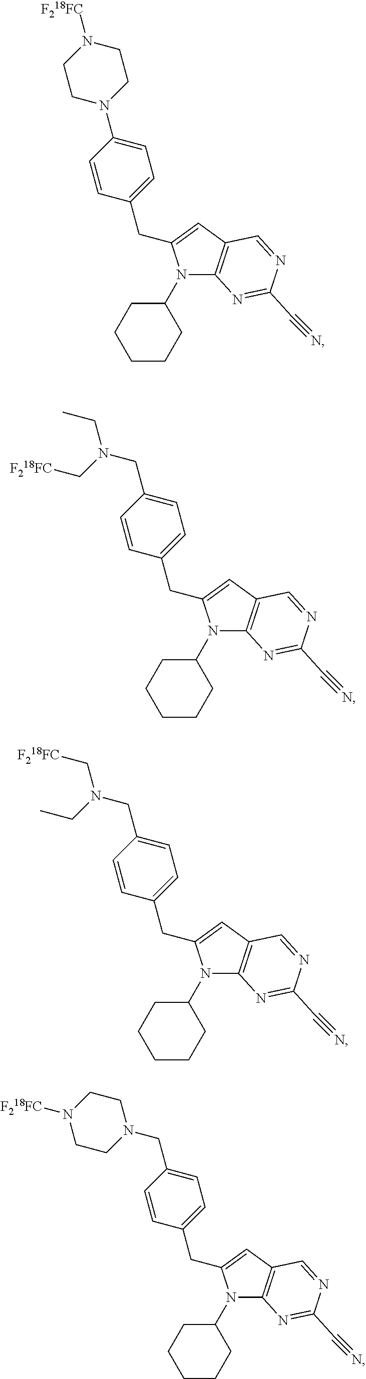

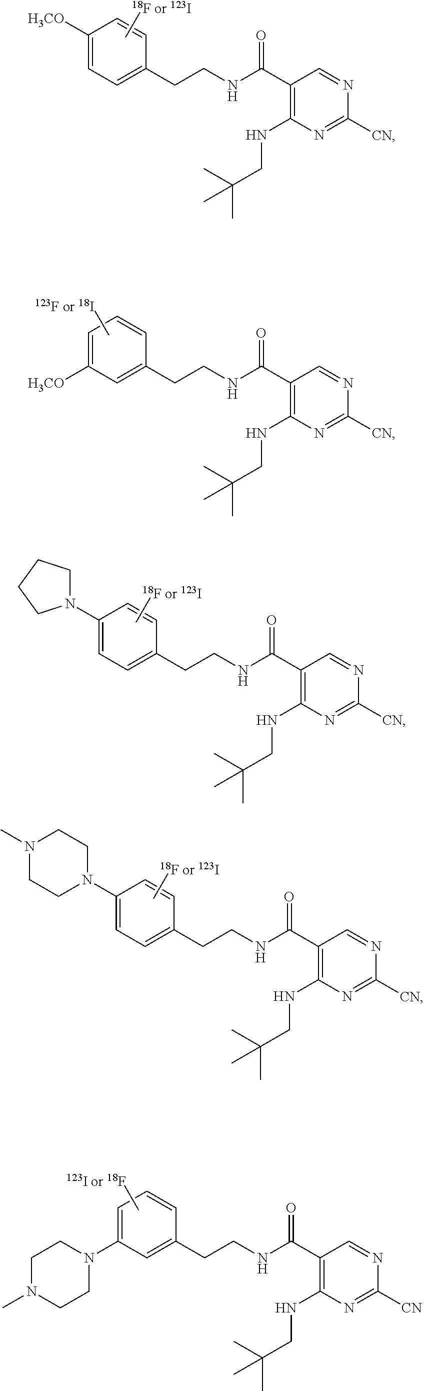

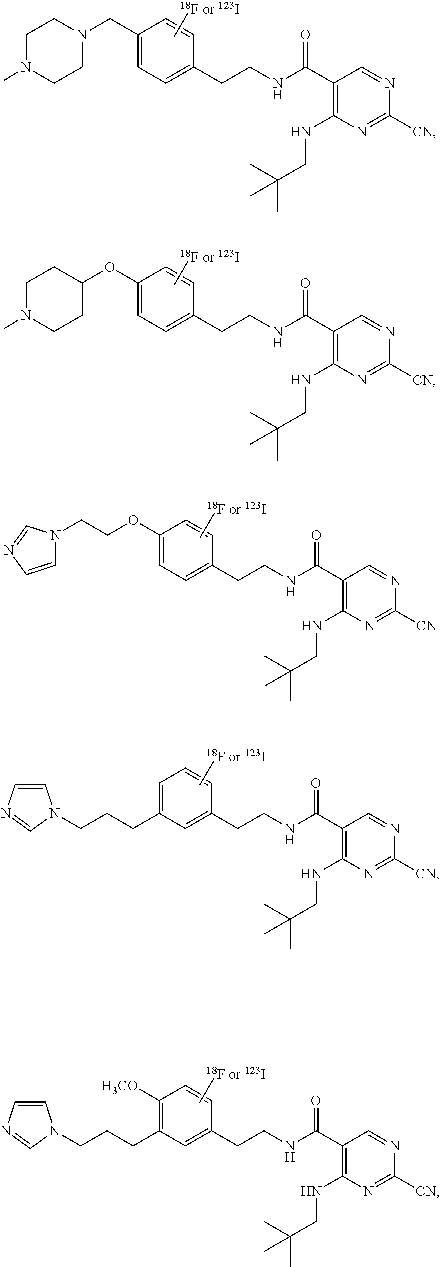

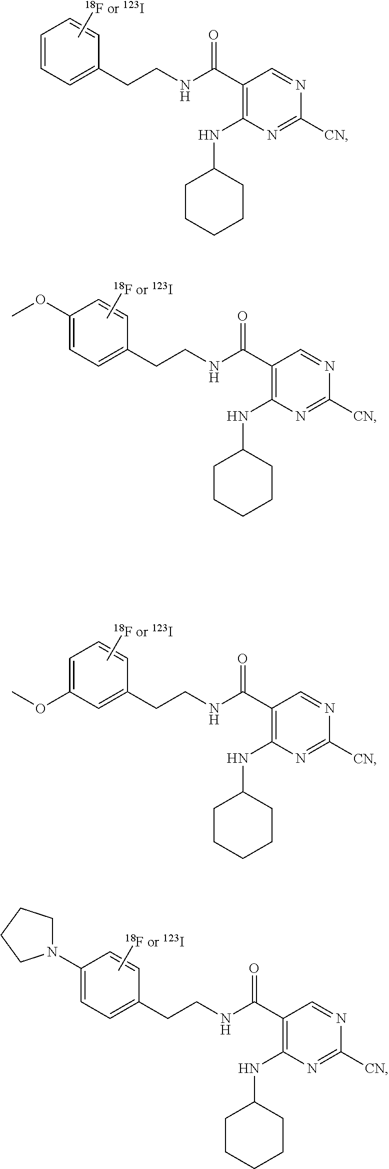

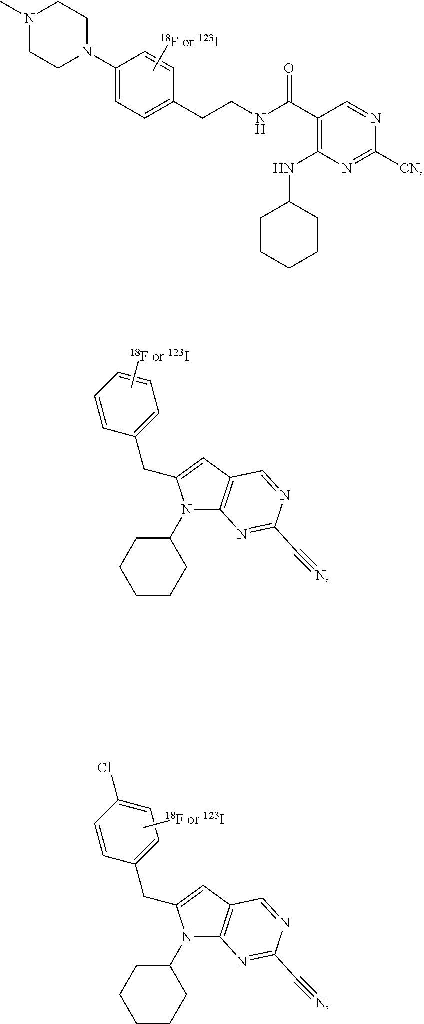

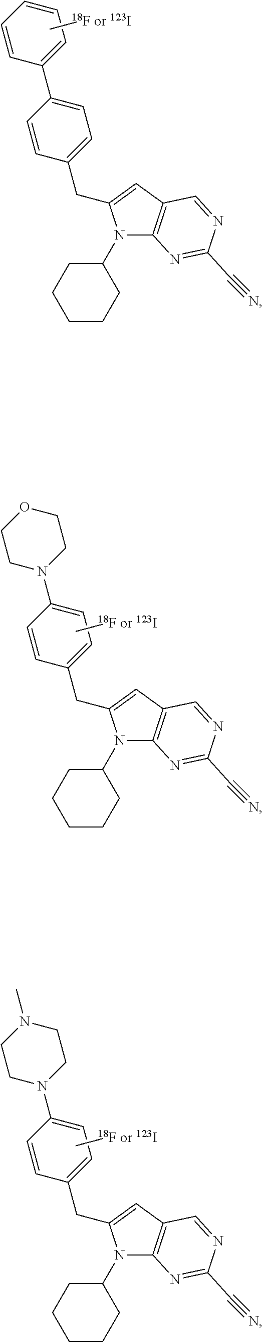

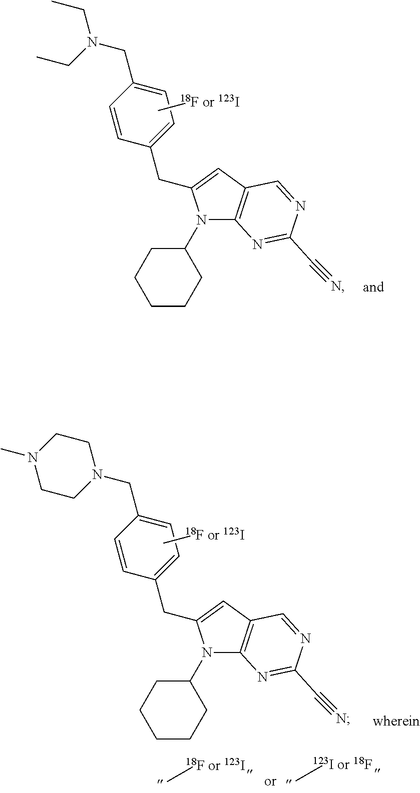

indicates that a Hydrogen molecule on the respective aromatic ring is replaced with either ¹⁸F or ¹²³I.

In certain embodiments, the present invention provides imaging compositions comprising a radiolabeled cathepsin K inhibitor of the present invention.

In certain embodiments, the present invention provides methods for imaging comprising the following steps (a) administering a radiolabeled cathepsin K inhibitor of the present invention to a subject; and (b) detecting gamma radiation emitted by the composition and forming an image therefrom. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for assessing osteoclast activity and/or bone mineral density (BMD) (e.g., rate of change of BMD) in a subject, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, and (d) assessing osteoclast activity and/or bone mineral density (BMD) (e.g., rate of change of BMD) in the subject based upon the quantifying of the amount of cathepsin K activity in the subject. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for assessing the presence, absence and/or potential for onset of osteoporosis in a subject, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with osteoporosis, and (f) determining the presence, absence and/or potential for onset of osteoporosis in the subject based upon the comparing. In some embodiments, the one or more established cathepsin K activity norms associated with osteoporosis are one or more selected from the group consisting of established cathepsin K activity norms for subjects having a pre-osteoporosis condition, established cathepsin K activity norms for subjects diagnosed with moderate osteoporosis, and established cathepsin K activity norms for subjects diagnosed with severe osteoporosis. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for generating a subject's risk profile for developing osteoporosis, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with osteoporosis, and (f) generating a subject's risk profile for osteoporosis based upon the comparing. In some embodiments, the one or more established cathepsin K activity norms associated with osteoporosis are one or more selected from the group consisting of established cathepsin K activity norms for subjects having a pre-osteoporosis condition, established cathepsin K activity norms for subjects diagnosed with moderate osteoporosis, established cathepsin K activity norms for subjects diagnosed with severe osteoporosis, established cathepsin K activity norms for subjects having a low risk for osteoporosis onset, established cathepsin K activity norms for subjects having a moderate risk for osteoporosis onset, and established cathepsin K activity norms for subjects having a high risk for osteoporosis onset. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for assessing the presence, absence and/or potential for onset of a musculoskeletal disorder (e.g., osteoarthritis, Rheumatoid arthritis, osteoporosis, osteopetrosis) associated with aberrant cathepsin K activity in a subject, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with a musculoskeletal disorder (e.g., osteoarthritis, Rheumatoid arthritis, osteoporosis, osteopetrosis) associated with aberrant cathepsin K activity, and (f) determining the presence, absence and/or potential for onset of such a musculoskeletal disorder associated with aberrant cathepsin K activity in the subject based upon the comparing. In some embodiments, the one or more established cathepsin K activity norms associated with a musculoskeletal disorder (e.g., osteoarthritis, Rheumatoid arthritis, osteoporosis, osteopetrosis) associated with aberrant cathepsin K activity are one or more selected from the group consisting of established cathepsin K activity norms for subjects having a particular musculoskeletal disorder (e.g., osteoarthritis, Rheumatoid arthritis, osteoporosis, osteopetrosis) associated with aberrant cathepsin K activity pre-osteoporosis condition, established cathepsin K activity norms for subjects diagnosed with a moderate severity for a musculoskeletal disorder (e.g., osteoarthritis, Rheumatoid arthritis, osteoporosis, osteopetrosis) associated with aberrant cathepsin K activity, established cathepsin K activity norms for subjects diagnosed with a severe case of a musculoskeletal disorder (e.g., osteoarthritis, Rheumatoid arthritis, osteoporosis, osteopetrosis) associated with aberrant cathepsin K activity, established cathepsin K activity norms for subjects having a low risk for musculoskeletal disorder (e.g., osteoarthritis, Rheumatoid arthritis, osteoporosis, osteopetrosis) associated with aberrant cathepsin K activity onset, established cathepsin K activity norms for subjects having a moderate risk for musculoskeletal disorder (e.g., osteoarthritis, Rheumatoid arthritis, osteoporosis, osteopetrosis) associated with aberrant cathepsin K activity onset, and established cathepsin K activity norms for subjects having a high risk for musculoskeletal disorder (e.g., osteoarthritis, Rheumatoid arthritis, osteoporosis, osteopetrosis) associated with aberrant cathepsin K activity onset. In some embodiments, the subject is any type of mammal. In some embodiments, the subject is a human being. In some embodiments, the imaging is PET imaging. In some embodiments, the imaging is SPECT imaging.

### BRIEF DESCRIPTION OF THE DRAWINGS

FIG. 1 shows structures of carbon-11 labeled cathepsin K inhibitors.

FIG. 2 shows summed microPET images (0-60 min) for (A) cathepsin K radioligand [¹¹C] 1 and (B) [¹⁸F]fluoride ion in normal rat (image greyscaled).

FIG. 3 shows representative tissue time vs. radioactivity curves for cathepsin K radioligand uptake and distribution in normal rat. Panel 3A: tissue kinetics in non-target regions of brain, heart, liver and muscle, and clearance into bladder; panel 3B: uptake and retention of radioactivity into selected target bone areas relative to non-specific distribution into muscle; panel 3C: reductions of bone uptake and retention after pre-administration of blocking dose (2 mg/kg) of unlabeled cathepsin K inhibitor; panel 3D: comparison of uptake and retention of cathepsin K radioligand in bone following intravenous injection of high and low specific activity preparations.

### DETAILED DESCRIPTION

Osteoporosis is estimated to affect over half of adults in the United States over the age of 50, and 1 in 3 women over the age of 80 will suffer a hip fracture; of those who fracture, nearly 15-20% die within 1 year. Overall, the costs associated with osteoporosis in the United States alone were estimated at $19 billion in 2005.

Currently, there are several ways to detect osteoporosis and other musculoskeletal diseases. First, conventional radiography can be used to measure bone mineral density (BMD) alone, or in conjunction with either CT or MRI scanning, to detect small fractures and reduced bone mass. However, these tests are insensitive and can only detect bone loss once it has progressed to an advanced stages, or fractures once they have occurred. Therefore, these tests are helpful only to determining a diagnosis. Second, Dual Energy X-ray Absorptiometry (e.g., DXA, or DEXA) is the most common test to determine BMD. This test is more sensitive than conventional radiography and reports a BMD as a T-score, which is compared to the T-score of a young health adult, and can be used for early detection as well as diagnosis. However, this test has been questioned as it compares results with peak bone mass of young adults (T-score) rather than that of a same aged control (Z-score). Third, Quantitative Computed Tomography (QCT) gives a separate reading for both trabecular and cortical bone, and reports BMD in mg/cm³ rather than a relative Z-score. Therefore, it improves on some of the problems with DXA. However, this method requires a high dose of radiation, compared to methods such as DXA, and results can be operator-dependent. Four, Quantitative Ultrasound uses the heel bone as a common skeletal site as it has a high percentage of trabecular bone. This is inexpensive, quick, easy to use, and does not require radiation. However, it is tarsal-specific and far more sensitive imaging machinery are required for this to be effective.

As such, there remains a need for a more sensitive and specific method to accurately detect changes in osteoclast activity and/or bone mineral density (BMD) (e.g., rate of change of BMD), as well as to detect early stage osteoporosis, before significant bone degradation can occur.

Cathepsin K is one of a family of cysteine peptidases with a primary physiological function of cleavage of type I and type II collagen (see, e.g., Novinec, M., et al., Biol Chem. 2013; 394:1163-1179). The enzyme is highly expressed in osteoclasts, multinucleated cells that are principally involved in resorption of bone in normal and pathological conditions. Deficiencies of cathepsin K cause a rare genetic disease, pycnodysotosis, whereas excessive function of osteoclasts (and expression of cathepsin K) produces bone loss and such diseases as osteoporosis (see, e.g., Troeberg, et al., Biochim Biophys Acta. 1824; 2012:133-145). The proteolytic activity of cathepsin K has also been demonstrated in numerous other diseases, including examples in oncology (see, e.g., Ishida, et al., J Eur Acad Dermatol Venereol. 2012; 27:128-130; Husmann, et al., Mol Carcinog. 2008; 47:66-73) and cardiovascular disease (see, e.g., Jaffer, et al., Circulation. 2007; 115:2292-2298).

A very large number of inhibitors of cathepsin K have now been synthesized and evaluated both in vitro and in vivo, with several compounds in current clinical trials (see, e.g., Stoch, et al., Nature. 2008; 83:172-176). In addition to the therapeutic potential of cathepsin K inhibitors, the high expression of the enzyme in osteoclasts may also provide a unique method for non-invasive imaging of biochemical changes in diseases involving up- or down-regulation of osteoclast numbers and/or activity. The potential of targeting cathepsin K was demonstrated both in vitro and in vivo using a near-infrared reporter probe attached to a cathepsin K activatable substrate (see, e.g., Kozloff, et al., Bone. 2009; 44:190-198), which provided an imaging method based on enzymatic activity.

Current clinical tools for non-invasive imaging of skeletal diseases include anatomical imaging methods, such as radiography, dual-energy x-ray absorptiometry, MRI and CT, but these methods lack sensitivity for detecting early biochemical or cellular changes that occur before significant bone losses. The standard nuclear medicine imaging method using γ-camera imaging of ^{99m}Tc-methylene diphosphonate (MDP) requires incorporation of the radiopharmaceutical into the bone via the action of osteoblasts, and is poor at detecting osteolytic lesions. Positron emission tomography (PET) imaging of [¹⁸F]fluoride ion incorporation into bone has developed as a potentially better imaging method than traditional MDP bone scans (see, e.g., Li, et al., Bone. 2012; 50:128-139), but the mechanism of incorporation of the radiotracer is non-specific and provides no biochemical information relating to cellular activities (osteoclasts or osteoblasts). Recently, research efforts into the development of osteoclast-specific imaging agents have targeted the aᵥb₃ integrin as a marker of cell numbers, with successful demonstration in rodents of localization of a copper-64 labeled integrin into areas of increased cellular responses including osteoclasts and inflammatory cells (see, e.g., Wadas, et al., J Nucl Med. 2009; 50:1873-1880).

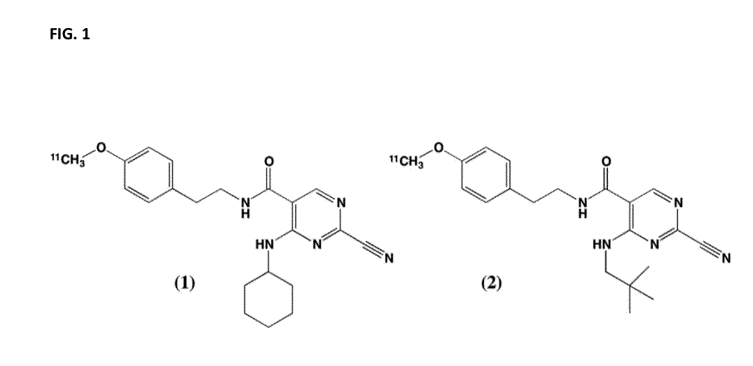

The present invention addresses such issues. The present invention provides molecular imaging radiotracers for nuclear medicine applications (e.g., PET imaging and SPECT imaging) for detecting and/or characterizing osteoporosis and other musculoskeletal diseases (e.g., osteoarthritis, rheumatoid arthritis, osteopetrosis) involving potential bone loss. Indeed, experiments conducted during the course of developing embodiments of the present invention developed carbon-11 labeling of two candidate high-affinity cathepsin K inhibitors (see, FIG. 1) and in vivo evaluation of the whole-body biodistribution and cathepsin K specific uptake into normal bone.

In particular, the present invention provides radiolabeled forms of known high affinity inhibitors of cathepsin K, a cysteine protease that is highly expressed in osteoclasts and that is responsible for type I collagen degradation and bone resorption. The potential for in vivo imaging of cathepsin K has been demonstrated using optical methods, and embodiments of the present invention extend this concept to a clinically translatable imaging method using radionuclide labeling. The present invention provides radiolabeled inhibitors based on cyanopyrimidine and pyrrolopyrimidine scaffolds, using isotopic (carbon-11) substitution or synthesis of fluorinated analogs (for fluorine-18 labeling) or iodinated analogs (for iodine-123 labeling). The compounds selected all have high affinity for human cathepsin K (<10 nanomolar), excellent selectivity over cathepsins L and S (100-1000 fold selective) and all are readily labeled using [11C]methyl iodide. New fluorinated analogs where fluoroethyl groups replace methyl groups are further provided. New iodinated analogs with aryl iodides are further provided.

In some embodiments, the present invention provides cathepsin K inhibitors based on cyanopyrimidine and/or pyrrolopyrimidine scaffolds (see, e.g., Altmann, et al., 2007 J. Med. Chem. Vol. 50(4) 591-594). For example, the present invention provides the following cathepsin K inhibitors:

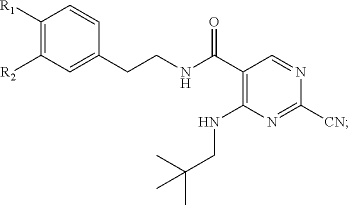

including salts, esters, and prodrugs thereof; and including both R and S enantiomeric forms and racemic mixtures thereof; wherein R1 is selected from CH₃O, H,

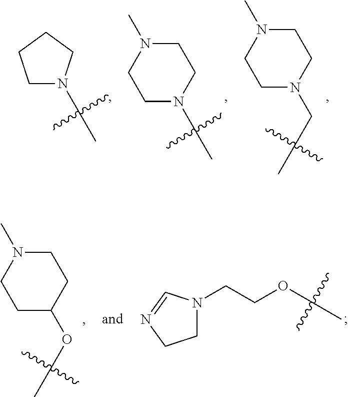

wherein R2 is selected from CH₃O, H,

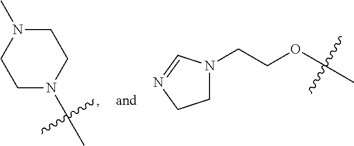

(see, e.g., Altmann, et al., 2007 J. Med. Chem. Vol. 50(4) 591-594). In some embodiments, the cathepsin K inhibitor is selected from

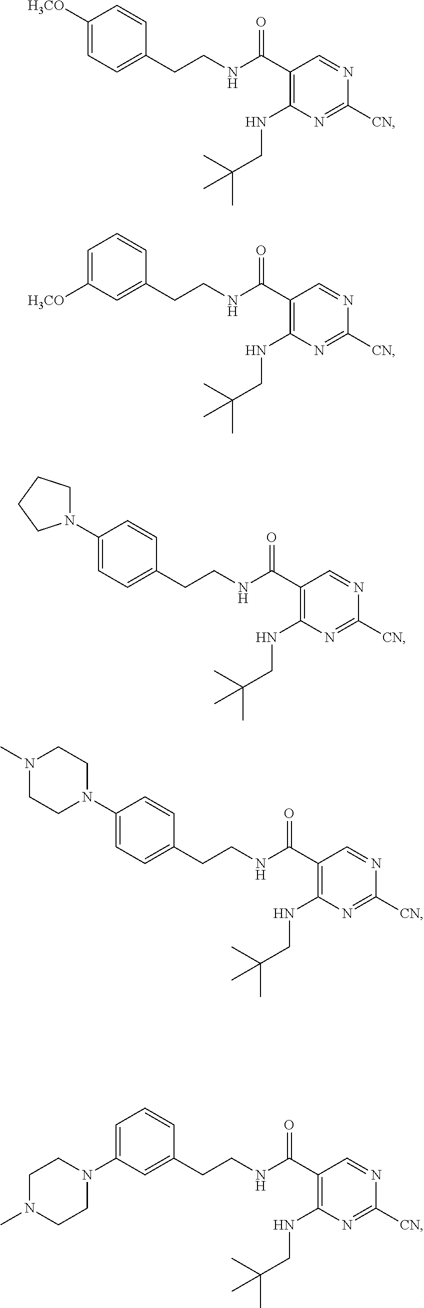 [SMILES: COc1ccc(CCNC(=O)c2cnc(C#N)nc2NCC(C)(C)C)cc1 ; COc1cccc(CCNC(=O)c2cnc(C#N)nc2NCC(C)(C)C)c1 ; CC(C)(C)CNc1nc(C#N)ncc1C(=O)NCCc1ccc(N2CCCC2)cc1 ; CN1CCN(c2ccc(CCNC(=O)c3cnc(C#N)nc3NCC(C)(C)C)cc2)CC1 ; CN1CCN(c2cccc(CCNC(=O)c3cnc(C#N)nc3NCC(C)(C)C)c2)CC1]

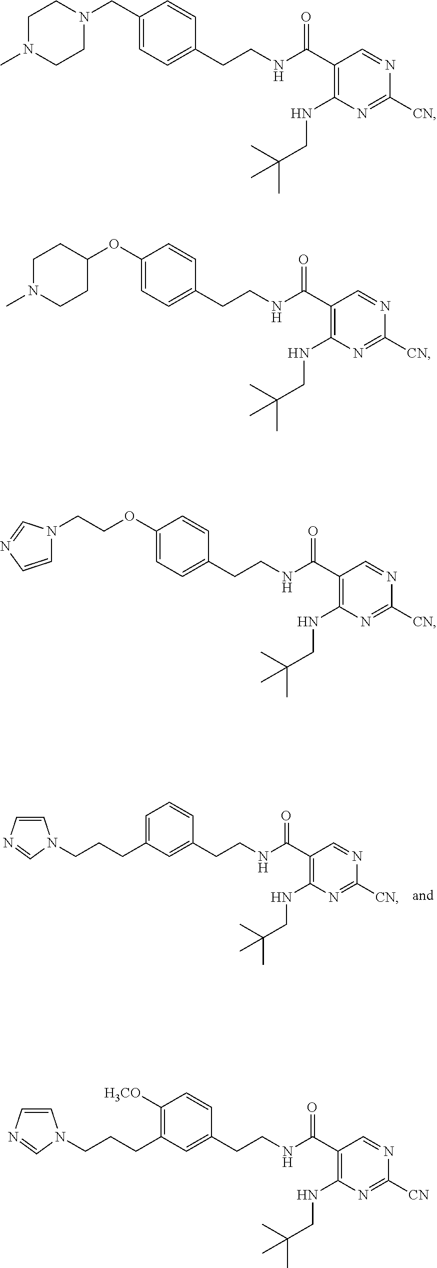 [SMILES: CN1CCN(Cc2ccc(CCNC(=O)c3cnc(C#N)nc3NCC(C)(C)C)cc2)CC1 ; CN1CCC(Oc2ccc(CCNC(=O)c3cnc(C#N)nc3NCC(C)(C)C)cc2)CC1 ; CC(C)(C)CNc1nc(C#N)ncc1C(=O)NCCc1ccc(OCCn2ccnc2)cc1 ; CC(C)(C)CNc1nc(C#N)ncc1C(=O)NCCc1cccc(CCCn2ccnc2)c1 ; COc1ccc(CCNC(=O)c2cnc(C#N)nc2NCC(C)(C)C)cc1CCCn1ccnc1]

(see, e.g., Altmann, et al., 2007 J. Med. Chem. Vol. 50(4) 591-594). In some embodiments, the cathepsin K inhibitor is selected from

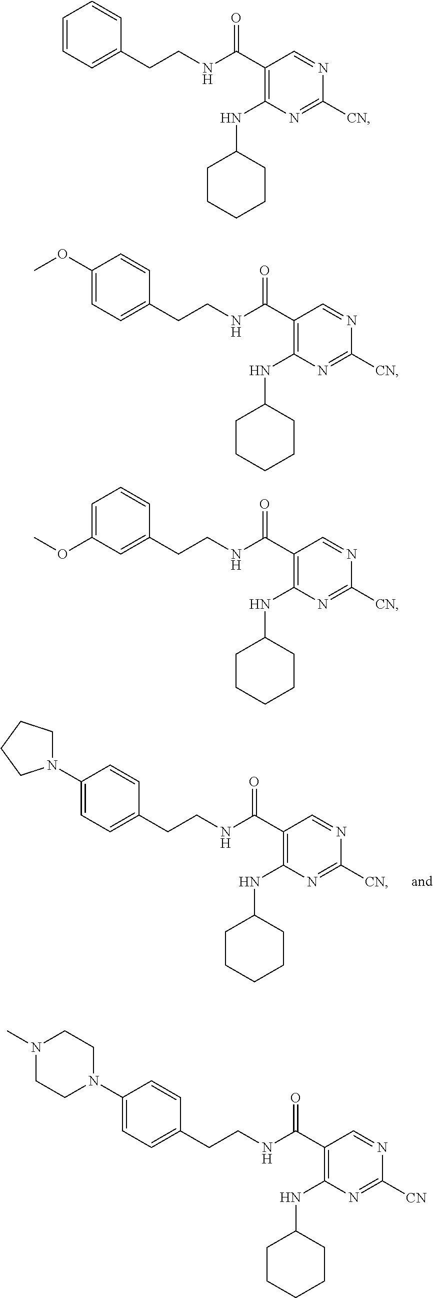

(see, e.g., Altmann, et al., 2007 J. Med. Chem. Vol. 50(4) 591-594).

The present invention further provides the following cathepsin K inhibitors:

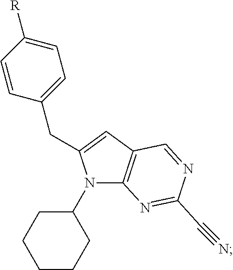

including salts, esters, and prodrugs thereof; and including both R and S enantiomeric forms and racemic mixtures thereof; wherein R is selected from H, Cl,

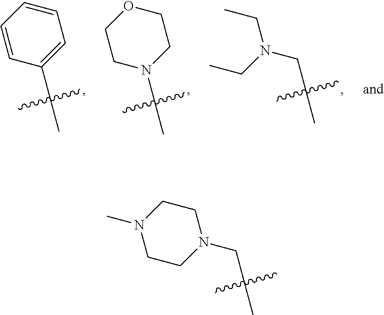

(see, e.g, Teno, et al., 2007 Bioorg. Med. Chem. Lett. 6096-6100). In some embodiments, the cathepsin K inhibitor is selected from

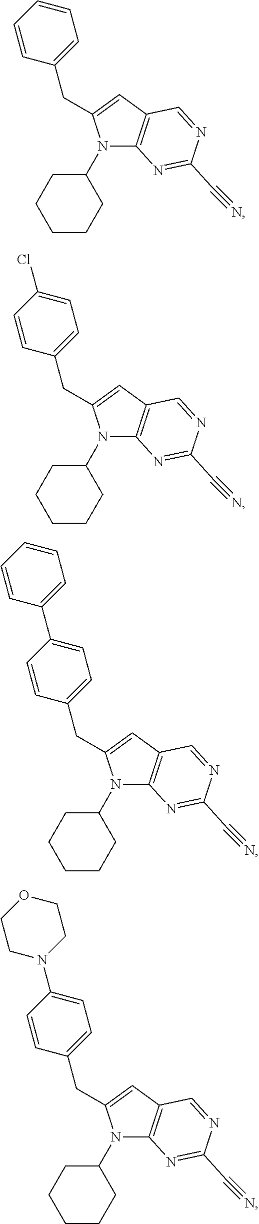

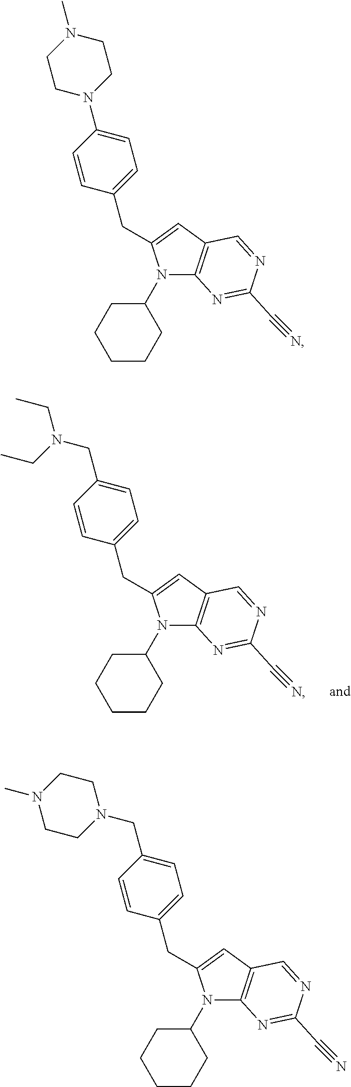 [SMILES: CN1CCN(c2ccc(Cc3cc4cnc(C#N)nc4n3C3CCCCC3)cc2)CC1 ; CCN(CC)Cc1ccc(Cc2cc3cnc(C#N)nc3n2C2CCCCC2)cc1 ; CN1CCN(Cc2ccc(Cc3cc4cnc(C#N)nc4n3C3CCCCC3)cc2)CC1]

(see, e.g, Teno, et al., 2007 Bioorg. Med. Chem. Lett. 6096-6100).

Additional examples of cathepsin K inhibitors include, but are not limited to,

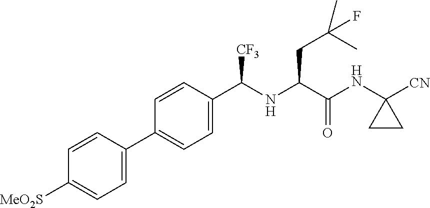 [SMILES: CC(C)(F)C[C@H](N[C@@H](c1ccc(-c2ccc(S(C)(=O)=O)cc2)cc1)C(F)(F)F)C(=O)NC1(C#N)CC1]

(odanacatib; N-(1-cyanocyclopropyl)-4-fluoro-N²-{(1S)-2,2,2-trifluoro-1-[4′-(methylsulfonyl)biphenyl-4-yl]ethyl}-L-leucinamide; Merck) (see, e.g., U.S. Pat. Nos. 8,318,748, 7,973,037, 7,928,091, 7,687,524, 7,605,159, 7,407,959, 7,405,229, 7,375,134, 7,279,478, 7,012,075, and 6,835,727),

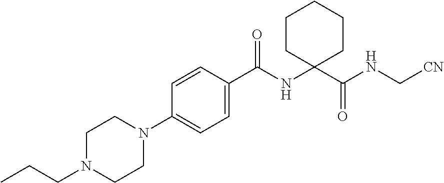 [SMILES: CCCN1CCN(c2ccc(C(=O)NC3(C(=O)NCC#N)CCCCC3)cc2)CC1]

(balicatib; Novartis),

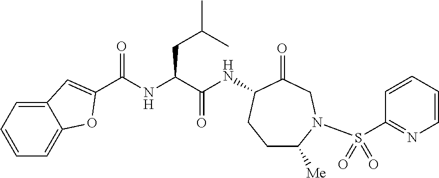 [SMILES: CC(C)C[C@H](NC(=O)c1cc2ccccc2o1)C(=O)N[C@H]1CC[C@@H](C)N(S(=O)(=O)c2ccccn2)CC1=O]

(relacatib; GlaxoSK),

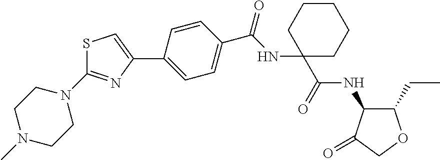 [SMILES: CC[C@@H]1OCC(=O)[C@H]1NC(=O)C1(NC(=O)c2ccc(-c3csc(N4CCN(C)CC4)n3)cc2)CCCCC1]

(MV061194; Medivir AB) (see, also, Bromme, 2009 Expert Opin. Invest. Drugs 18(5), 585-600),

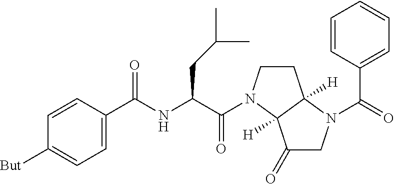

(Amura) (see, also, Bromme, 2009 Expert Opin. Invest. Drugs 18(5), 585-600), and denosumab (Amgen); see, also, e.g., U.S. Patent Application Publication No. 2008/0267887, 2009/0318441; Stoch, et al., 2008 Clinical Pharmacology & Therapeutics 83 172-176.

As described above, such cathepsin K inhibitor compounds are radio-labeled for imaging purposes. In some preferred embodiments, the radio-label is selected from the group consisting of ¹¹C, ¹⁸F and ¹²³I. In some embodiments, all stereoisomers for each described compound are contemplated. The present invention is not limited to a particular manner of radiolabeling such cathepsin K inhibitors.

In some embodiments, the cathepsin K inhibitor compounds are radiolabelled as shown in FIG. 1.

In some embodiments, the cathepsin K inhibitor compounds are radiolabelled in the following manner:

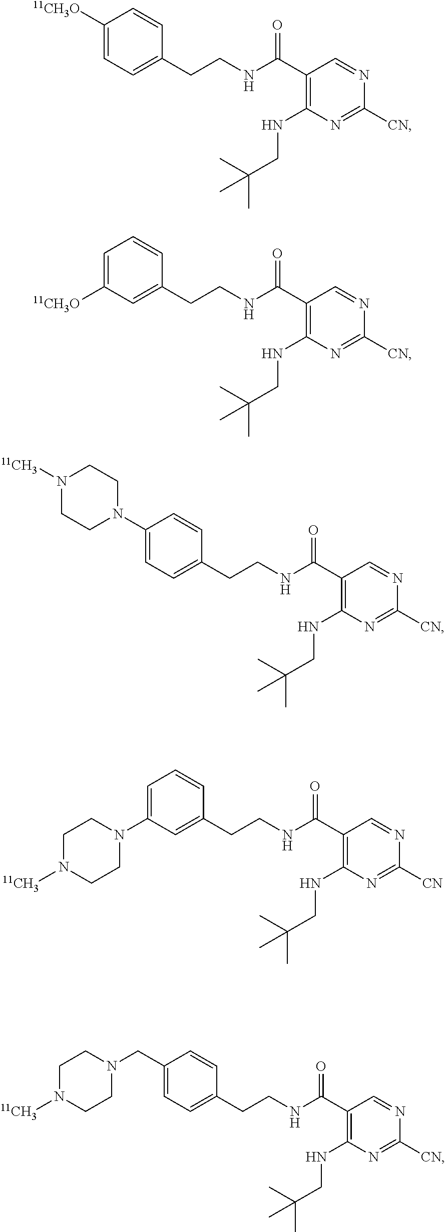

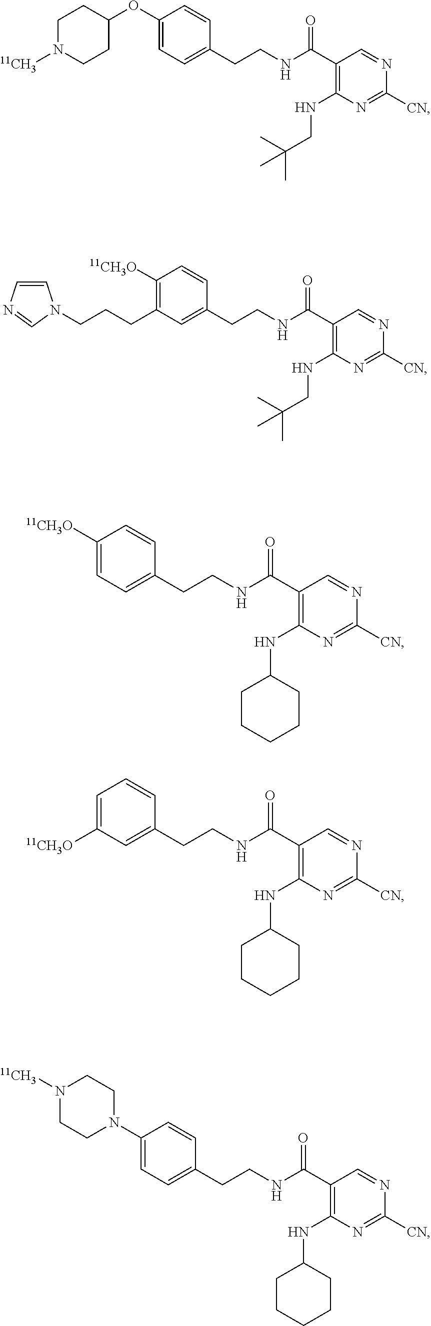

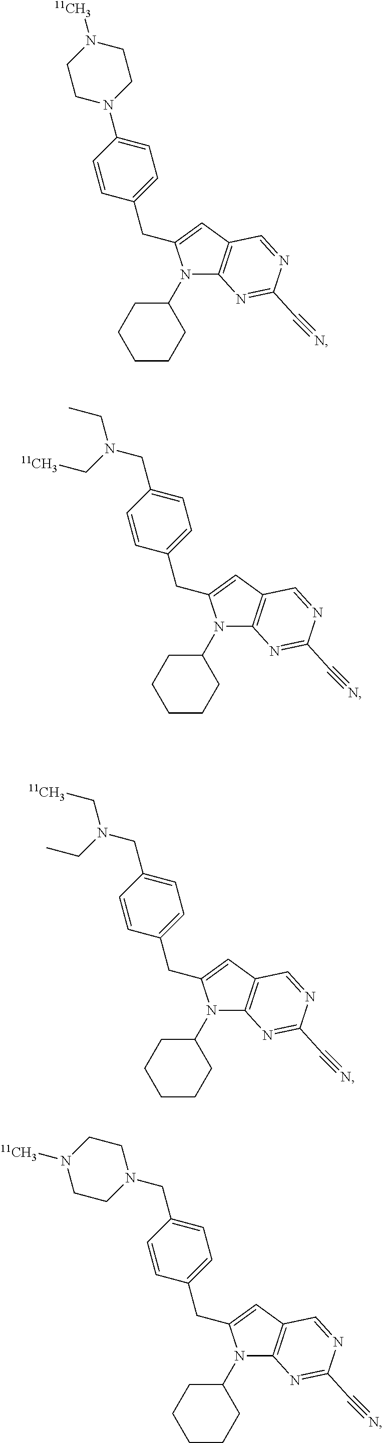 [SMILES: [11CH3]N1CCN(c2ccc(Cc3cc4cnc(C#N)nc4n3C3CCCCC3)cc2)CC1 ; CCN(C[11CH3])Cc1ccc(Cc2cc3cnc(C#N)nc3n2C2CCCCC2)cc1 ; CCN(C[11CH3])Cc1ccc(Cc2cc3cnc(C#N)nc3n2C2CCCCC2)cc1 ; [11CH3]N1CCN(Cc2ccc(Cc3cc4cnc(C#N)nc4n3C3CCCCC3)cc2)CC1]

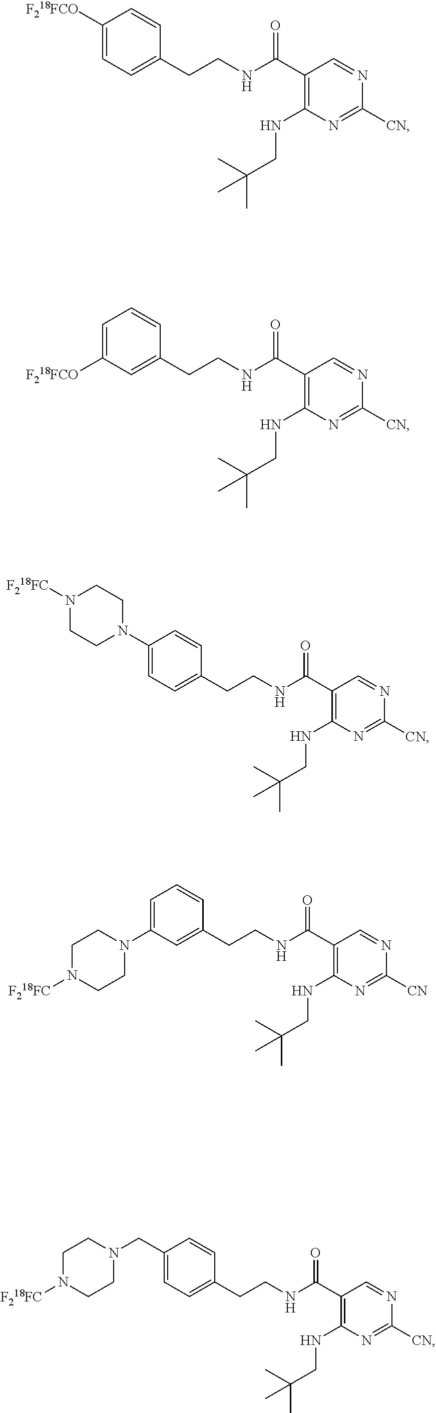

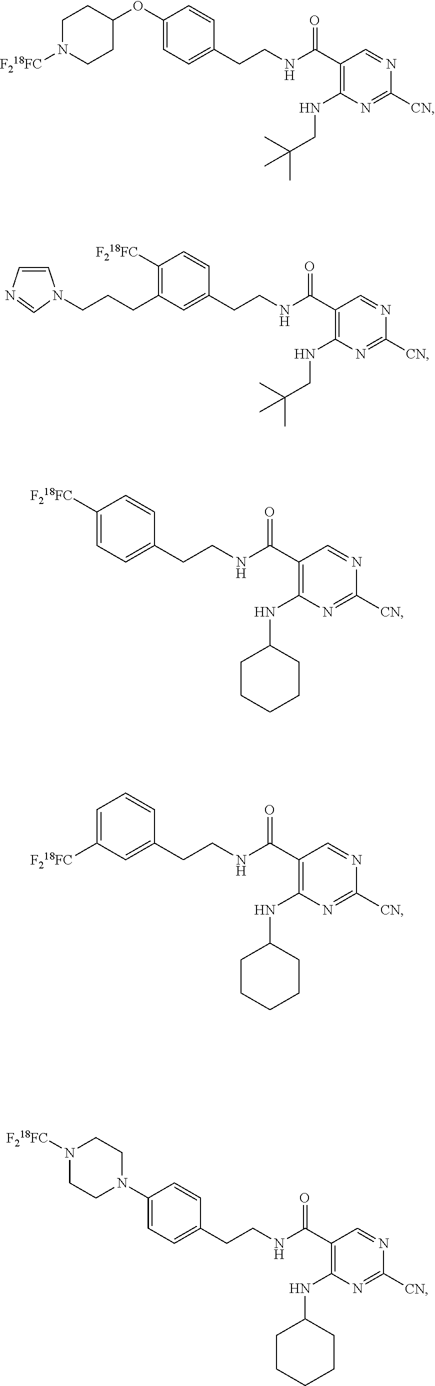

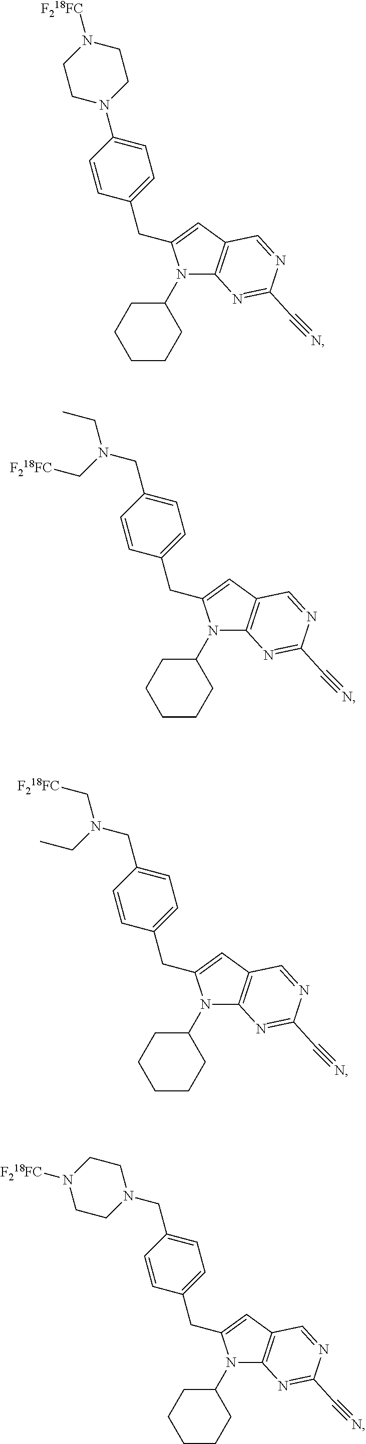

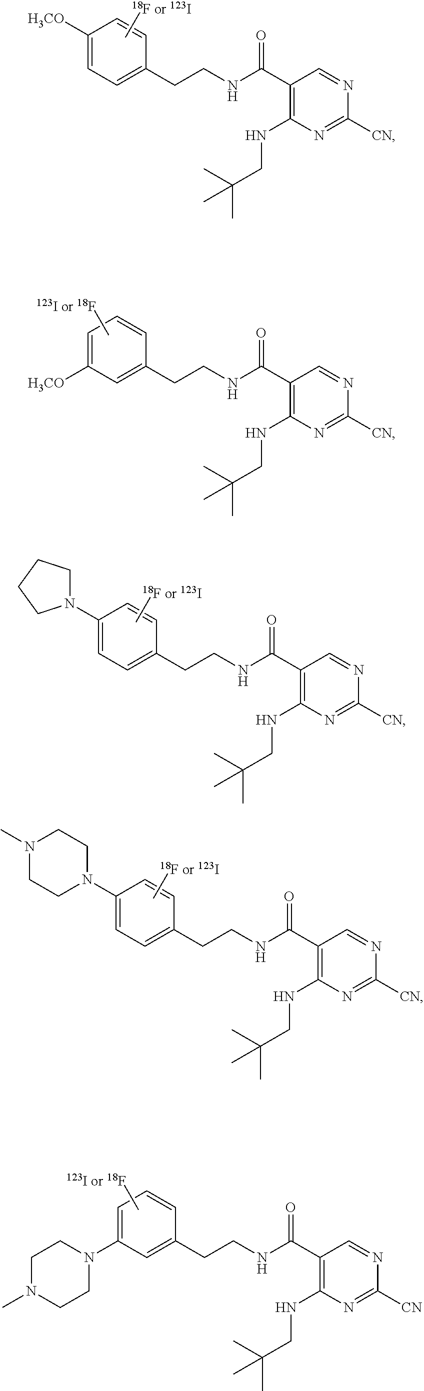

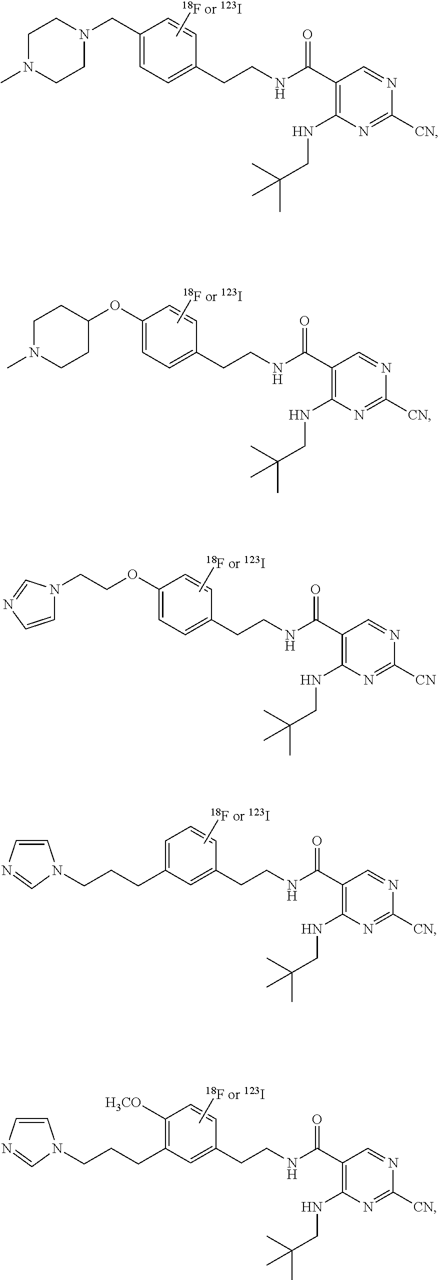

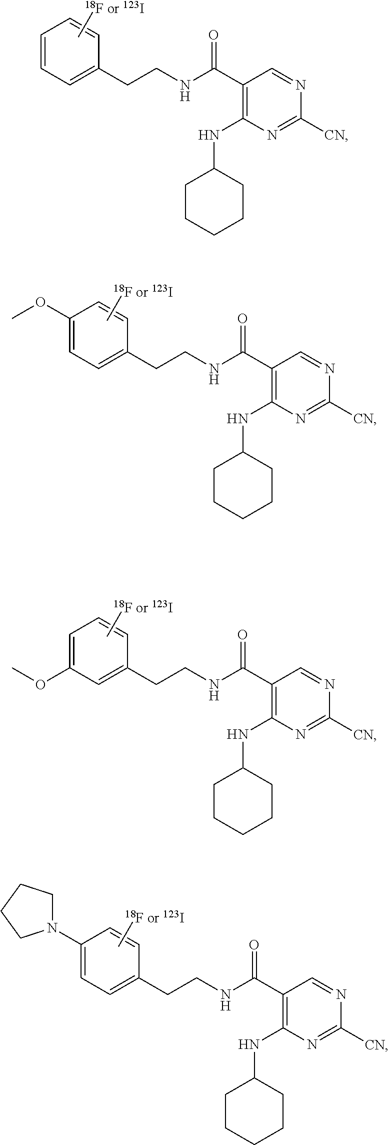

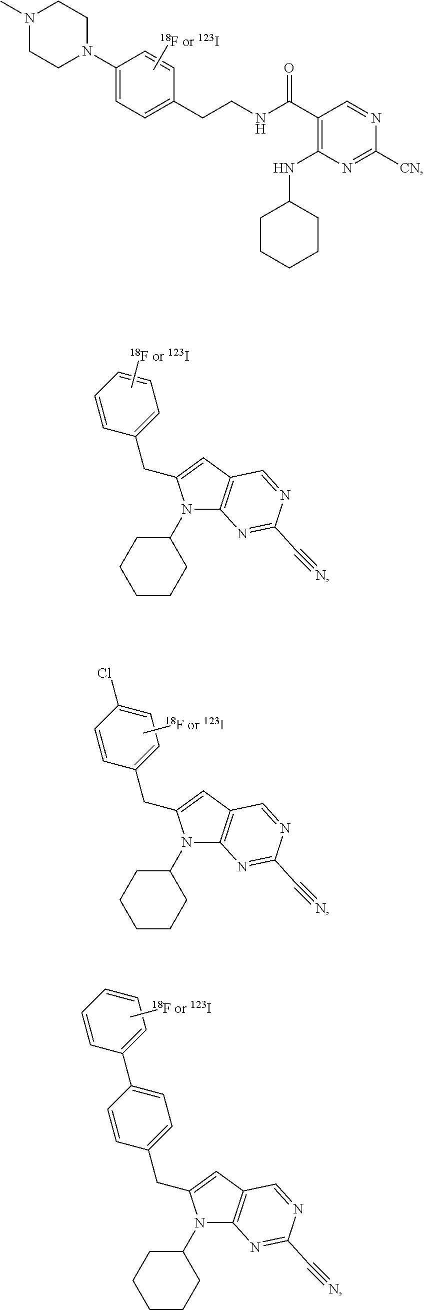

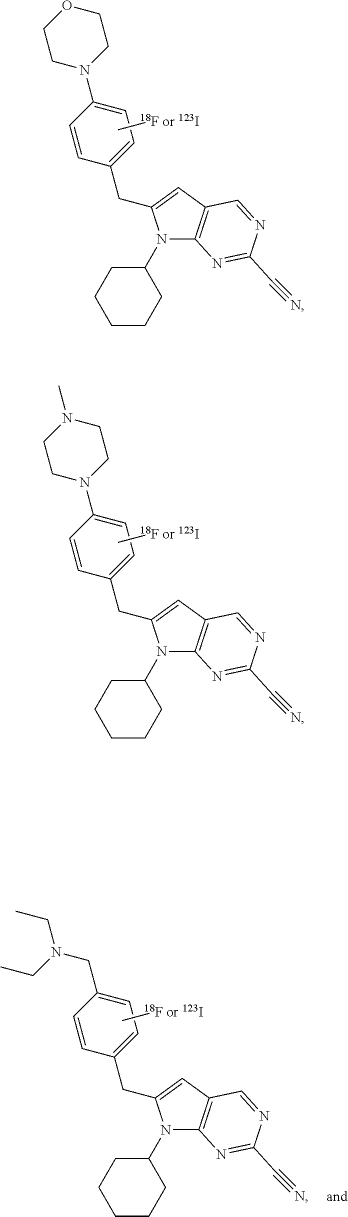

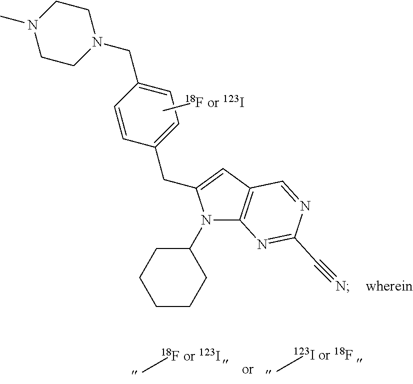

indicates that a Hydrogen molecule on the respective aromatic ring is replaced with either ¹⁸F or ¹²³I.

The radiolabeled cathepsin K inhibitors of the present invention find many uses. In particular, the radiolabeled cathepsin K inhibitors of the present invention find use as imaging agents within nuclear medicine imaging protocols (e.g., PET imaging, SPECT imaging).

PET is the study and visualization of human physiology by electronic detection of short-lived positron emitting radiopharmaceuticals. It is a non-invasive technology that quantitatively measures metabolic, biochemical and functional activity in living tissue.

The PET scan is a vital method of measuring body function and guiding disease treatment. It assesses changes in the function, circulation and metabolism of body organs. Unlike MRI (Magnetic Resonance Imaging) or CT (Computed Tomography) scans which primarily provide images of organ anatomy, PET measures chemical changes that occur before visible signs of disease are present on CT and MRI images.

PET visualizes behaviors of trace substances within a subject (e.g., a living body) having a radioimaging agent administered therein by detecting a pair of photons occurring as an electron/positron pair annihilation and flying in directions opposite from each other (see, e.g., U.S. Pat. No. 6,674,083; herein incorporated by reference in its entirety).

Single Photon Emission Computed Tomography (SPECT) is a tomographic nuclear imaging technique producing cross-sectional images from gamma ray emitting radiopharmaceuticals (single photon emitters or positron emitters). SPECT is routinely used in clinical studies. SPECT is performed by using a gamma camera, comprising a collimator fixed on a gamma detector, which gamma camera follows a revolution orbit around the patient's body. The gamma rays, emitted by a radioactive tracer, accumulated in certain tissues or organs of the patient's body, are sorted by the collimator and recorded by the gamma detector under various angles around the body, the collimator always pointing to (facing) the rotation axis of the camera. From the acquired planar images the distribution of the activity inside the patient's body can be computed using certain reconstruction algorithms.

In some embodiments, the radiolabeled cathepsin K inhibitors of the present invention are used with Positron Emission Tomography (PET). PET involves the detection of gamma rays in the form of annihilation photons from short-lived positron emitting radioactive isotopes including, but not limited to ¹⁸F with a half-life of approximately 110 minutes, ¹¹C with a half-life of approximately 20 minutes, ¹³N with a half-life of approximately 10 minutes and ¹⁵O with a half-life of approximately 2 minutes, using the coincidence method. In some embodiments, the radiolabeled cathepsin K inhibitors of the present invention are used with PET to produce a 3-dimentional image of a subject's osteoclast bone activity. In some embodiments, the radiolabeled cathepsin K inhibitors of the present invention are used with single-photon emission computed tomography (SPECT). SPECT involves the detection of gamma rays from isotopes including but not limited to iodine-123 with a half-life of 13.3 hours.

The radiolabeled cathepsin K inhibitors of the present invention are not limited to particular uses. In some embodiments, the radiolabeled cathepsin K inhibitors of the present invention are used as a biomarker for any musculoskeletal disease (e.g., osteoarthritis, rheumatoid arthritis, osteoporosis, osteopetrosis) involving bone loss. In certain embodiments, the radiolabeled cathepsin K inhibitors, through binding cathepsin K, are used as biomarkers for osteoclast activation. In some embodiments, such radiolabeled cathepsin K inhibitors are used as a biomarker for osteoporosis (including potential for onset of osteoporosis), prior to actual bone degradation. The ability to determine osteoclast activity (e.g., through binding and imaging of cathepsin K), prior to the breakdown of bone, render such radiolabeled cathepsin K inhibitors of the present invention more sensitive markers than those currently utilized in the art.

In some embodiments, the radiolabeled cathepsin K inhibitors of the present invention are used to diagnose bone activity that can lead to osteoporosis, prior to significant bone degradation. In some embodiments, the radiolabeled cathepsin K inhibitors of the present invention are used to diagnose bone activity consistent with various stages of osteoporosis (e.g., pre-osteoporosis, moderate osteoporosis, severe osteoporosis).

In some embodiments, the radiolabeled cathepsin K inhibitors of the present invention are used to detect a decrease in osteoclastic activity which is then used to investigate hormones and cytokines that inhibit osteoclasts. In some embodiments, the radiolabeled cathepsin K inhibitors of the present invention are used to detect a decrease in osteoclastic activity which is then used to investigate endocrine disorders that can induce bone loss (e.g., Cushing's syndrome, hyperparathyroidism, thyrotoxins, hypothyroidism, diabetes mellitus type 1 and 2, acoromegaly, and adrenal insufficiency).

In some embodiments, the radiolabeled cathepsin K inhibitors of the present invention are used to detect a decrease in osteoclastic activity which is then used to investigate inherited disorders linked to osteoporosis (e.g., osteogenesis imperfecta, Marfansyndrome, hemochromatosis, hypophosphatasia, glycogen storage diseases, homocystinuria, Ehlers-Danlos syndrome, porphyria, Menkes' syndrome, epidermolysis bullosa and Gaucher's disease). For example, such radiolabeled cathepsin K inhibitors of the present invention are used to determine the onset of osteoporosis in such inherited disorders linked to osteoporosis or might be sensitive enough to diagnose the onset of the disease.

In certain embodiments, the present invention provides methods for assessing the presence or absence of osteoporosis in a subject, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with osteoporosis, and (f) determining the presence or absence of osteoporosis in the subject based upon the comparing. In some embodiments, the one or more established cathepsin K activity norms associated with osteoporosis are one or more selected from the group consisting of established cathepsin K activity norms for subjects having a pre-osteoporosis condition, established cathepsin K activity norms for subjects diagnosed with moderate osteoporosis, and established cathepsin K activity norms for subjects diagnosed with severe osteoporosis. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for generating a subject's risk profile for developing osteoporosis, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with osteoporosis, and (f) generating a subject's risk profile for osteoporosis based upon the comparing. In some embodiments, the one or more established cathepsin K activity norms associated with osteoporosis are one or more selected from the group consisting of established cathepsin K activity norms for subjects having a pre-osteoporosis condition, established cathepsin K activity norms for subjects diagnosed with moderate osteoporosis, established cathepsin K activity norms for subjects diagnosed with severe osteoporosis, established cathepsin K activity norms for subjects having a low risk for osteoporosis onset, established cathepsin K activity norms for subjects having a moderate risk for osteoporosis onset, and established cathepsin K activity norms for subjects having a high risk for osteoporosis onset. In some embodiments, the subject is any type of mammal. In some embodiments, the subject is a human being. In some embodiments, the imaging is PET imaging. In some embodiments, the imaging is SPECT imaging.

In certain embodiments, the present invention provides methods for assessing the presence or absence of a musculoskeletal disorder (e.g., osteoarthritis, Rheumatoid arthritis, osteoporosis, osteopetrosis) associated with aberrant cathepsin K activity in a subject, comprising the following steps (a) administering a composition comprising a radiolabeled cathepsin K inhibitor of the present invention to a subject; (b) detecting gamma radiation emitted by the composition and forming an image therefrom; (c) quantifying the amount of cathepsin K activity in the subject based upon the detecting and forming of an image, (d) assessing osteoclast activity in the subject based upon the quantifying of the amount of cathepsin K activity in the subject, (e) comparing the quantified cathepsin K activity with one or more established cathepsin K activity norms associated with a musculoskeletal disorder (e.g., osteoarthritis, Rheumatoid arthritis, osteoporosis, osteopetrosis) associated with aberrant cathepsin K activity, and (f) determining the presence or absence of such a musculoskeletal disorder associated with aberrant cathepsin K activity in the subject based upon the comparing. In some embodiments, the one or more established cathepsin K activity norms associated with a musculoskeletal disorder (e.g., osteoarthritis, Rheumatoid arthritis, osteoporosis, osteopetrosis) associated with aberrant cathepsin K activity are one or more selected from the group consisting of established cathepsin K activity norms for subjects having a particular musculoskeletal disorder (e.g., osteoarthritis, Rheumatoid arthritis, osteoporosis, osteopetrosis) associated with aberrant cathepsin K activity pre-osteoporosis condition, established cathepsin K activity norms for subjects diagnosed with a moderate severity for a musculoskeletal disorder (e.g., osteoarthritis, Rheumatoid arthritis, osteoporosis, osteopetrosis) associated with aberrant cathepsin K activity, established cathepsin K activity norms for subjects diagnosed with a severe case of a musculoskeletal disorder (e.g., osteoarthritis, Rheumatoid arthritis, osteoporosis, osteopetrosis) associated with aberrant cathepsin K activity, established cathepsin K activity norms for subjects having a low risk for musculoskeletal disorder (e.g., osteoarthritis, Rheumatoid arthritis, osteoporosis, osteopetrosis) associated with aberrant cathepsin K activity onset, established cathepsin K activity norms for subjects having a moderate risk for musculoskeletal disorder (e.g., osteoarthritis, Rheumatoid arthritis, osteoporosis, osteopetrosis) associated with aberrant cathepsin K activity onset, and established cathepsin K activity norms for subjects having a high risk for musculoskeletal disorder (e.g., osteoarthritis, Rheumatoid arthritis, osteoporosis, osteopetrosis) associated with aberrant cathepsin K activity onset.

In some embodiments, the subject is any type of mammal. In some embodiments, the subject is a human being. In some embodiments, the imaging is PET imaging. In some embodiments, the imaging is SPECT imaging.

In some embodiments, the radiolabeled cathepsin K inhibitors of the present invention are used to diagnose bone activity consistent with developing musculoskeletal diseases (e.g., osteoarthritis, rheumatoid arthritis, or osteoporosis) involving bone loss. In some embodiments, information obtained through the use of the radiolabeled cathepsin K inhibitors are compared with established norms for bone activity consistent with particular stages of musculoskeletal diseases (e.g., osteoarthritis, rheumatoid arthritis, or osteoporosis) (e.g., established cathepsin K activity norms for pre-osteoporosis condition, established cathepsin K activity norms for subjects diagnosed with moderate osteoporosis, and established cathepsin K activity norms for subjects diagnosed with severe osteoporosis) thereby facilitating a respective diagnosis and/or assessment of risk of developing such a disease. In some embodiments, information obtained through the use of the radiolabeled cathepsin K inhibitors (e.g., diagnoses of a particular stage of a musculoskeletal disease (e.g., osteoarthritis, rheumatoid arthritis, or osteoporosis) and/or assessment of risk of developing such a disease) is used in developing and implementing a treatment program and/or preventive treatment program.

In some embodiments, fluorinated analogs where fluoroethyl groups are used to replace methyl groups are provided. In some embodiments, aryl iodides are incorporated. In some embodiments, such fluorinated or iodinated analogs will be evaluated in vitro for inhibitory action on cathepsin K enzymatic activity, using a fluorescent assay, and potent (<10 nM) inhibitors labeled using analogous [¹⁸F]fluoroalkylation or [¹²³I]iodination reactions.

In vivo proof of concept studies to demonstrate osteoclast-dependent localization of radiotracers will be done in rats using focal skeletal injections of RANKL (receptor activator for nuclear factor κ B ligand, to induce osteoclastogenesis) or osteoprotogerin (to inhibit osteoclastogenesis). Extent of bone loss will be monitored by microCT. Verification that radioactivity localization is cathepsin-specific will be done in RANKL treated rats and blocking with cold doses of enzyme inhibitor. Successful radiotracers that exhibit high uptake and retention in RANKL-treated animals that is blocked by cold inhibitor, and reduced uptake in osteoprotegrin-treated animals, will then be further developed as potential radioligands for human imaging. It is expected that detection of increased osteoclastic cellular activity will provide new diagnostic criteria that take osteoclast activity into account in addition to standard measures of structural integrity, and thus provide immediate feedback on the efficacy of a chosen treatment protocol before waiting for further downstream gain or loss of bone to occur.

It is contemplated that the radiolabeled cathepsin K inhibitors of the present invention are provided to a nuclear pharmacist or a clinician in kit form. In accordance, the radiolabeled cathepsin K inhibitors of the present invention can be readily made by a clinician or pharmacist at the location of intended use, thus avoiding shipment of a premade radioiodinated compound.

A pharmaceutical composition of the present invention comprises one of the aforementioned radiolabeled cathepsin K inhibitors and a carrier such as a physiological buffered saline solution or a physiologically buffered sodium acetate carrier. It is contemplated that the composition will be systemically administered to a subject as by intravenous injection.

It will be appreciated by those skilled in the art that the radiolabeled cathepsin K inhibitors of the present invention are employed in accordance with conventional methodology in nuclear medicine. Thus, a composition of the present invention is typically systemically applied to a subject, and subsequently the uptake of the composition in the selected body region is measured and an image formed, for example, by means of a conventional gamma camera.

### EXPERIMENTAL

### Example I

Cathepsin K, a cysteine peptidase located in the osteoclast, is involved in bone resorption and excessive activity of this enzyme has been linked to osteoporosis. Imaging agents for Cat K might serve as indicators of osteoclast upregulation prior to significant detectable bone loss, providing earlier diagnosis of disease and potential feedback on treatment. The purpose of this study was the synthesis of 11C-labeled cathepsin K inhibitors (see, e.g., J Med Chem 2007:50; 591) for in vivo imaging of osteoclast concentrations.

Desmethyl precursors, 2-cyano-4-(cyclohexylamino)-N-(4-hydroxyphenethyl)pyrimidine-5-carboxamide and 2-cyano-N-(4-hydroxyphenethyl)-4-(neopentylamino)pyrimidine-5-carboxamide were synthesized following literature procedures. Syntheses of 2-cyano-4-(cyclohexylamino)-N-(4-[¹¹C]methoxyphenethyl)pyrimidine-5-carboxamide ([¹¹C]CCX, IC50 =0.0222 nM) and 2-cyano-N-(4-[¹¹C]methoxy-phenethyl)-4-(neopentylamino)pyrimidine-5-carboxamide ([¹¹C]CNP, IC50=0.003 nM) were done with no-carrier-added [¹¹C]MeI, using an automated radiosynthesis platform. Biodistribution studies were performed in mice. Dynamic microPET imaging was performed in rats.

Desmethyl precursors were synthesized in moderate yields (10-20%) and high chemical purities (>90%). [¹¹C]CCX and [¹¹C]CNP were synthesized in 10-15% yields (decay corrected), >95% chemical and >99% radiochemical purities, and specific activities >10 Ci/μmol. Biodistribution studies showed low overall uptake of radioactivity in normal tissues, including bone, and rapid clearance. MicroPET imaging indicated high uptake of [¹¹C]CCX in the growth plates located at the proximal humerus, distal ulnar, carpal, distal femur, proximal tibia, and caudal vertebrae. Uptake and retention of [¹¹C]CCX was reduced by pretreatment with unlabeled CCX.

[¹¹C]CCX and [¹¹C]CNP are the first cathepsin K inhibitors labeled with a PET radionuclide for in vivo imaging use. Increased uptake of [¹¹C]CCX in expected regions of high osteoclastic activity of normal bone, and low uptake in mature bone and soft tissues, supports further study of cathepsin K radioligands in animal models of osteoclastogenesis.

### Example II

This example describes general information, chemistry, radiochemical syntheses, and biology (e.g., materials and methods) for Example III.

### General

Organic and inorganic reagents and solvents (ACS grade) were purchased from commercially available sources and used without purification. Authentic unlabeled samples of compounds 1 and 2 were prepared by minor modifications of the published procedures (see, e.g., Altlman, J Med Chem. 2007; 50:591-594). Thin layer chromatography (TLC) was performed on glass plates pre-coated with 0.25 mm thickness of silica gel (60 Å) with fluorescent indicator (EMD). Automated flash chromatography was performed using a Biotage SP1 system with Isolute silica cartridges (Biotage). All ¹H and ¹³C NMR spectra were obtained using a Varian 400 MHz Mercury plus instrument at ambient temperatures in chloroform-d (CDCl₃) or methanol-d (CD₃OD). High resolution mass spectra were obtained on a Micromass Autospec in the ESI-TOF mode. Analytical (Luna C8(2) 100×2 mm) and semi-preparative (Luna C18 150×10 mm) HPLC columns were obtained from Phenomenex (Phenomenex). Radiochemical syntheses were done using a General Electric Medical Systems TRACERLab FX C Pro synthesis system. For radiochemical syntheses, Sep-Pak C18 1 cc cartridges were purchased from Waters and conditioned with ethanol (10 mL) and water (10 mL) prior to use.

### Chemistry

N-(4-(benzyloxy)phenethyl)-2,4-dichloropyrimidine-5-carboxamide (5b)

To a solution of 2-(4-(benzyloxy)phenyl)ethanamine hydrochloride (3.55 mmol) and diisopropylethylamine (7.46 mmol) in 10 ml of dichloromethane was added 2,4-dichloropyrimidine-5-carbonyl chloride (3.55 mmol), and the resulting suspension was stirred at ambient temperature for 24 h. The solvent was removed under reduced pressure, and the resulting oil was purified using flash chromatography on silica gel (dichloromethane/methanol 0-5%) to afford 5b (0.870 g, 61%) as a white solid. ¹H NMR (400 MHz, CDCl₃) δ 8.89 (s, 1H), 7.35-7.19 (m, 5H), 6.41 (broad s, 1H), 3.78 (dt, 2H, J=7.5, 6.3, Hz), 2.95 (t, 2H, J=6.3Hz). ¹³C NMR (101 MHz, CDCl₃) δ 161.51 (1C), 161.23 (1C), 158.27 (1C), 157.73 (1C), 136.83 (1C), 130.18 (1C), 129.72 (2C), 128.57 (2C), 127.98 (1C), 127.40 (2C), 126.97 (1C), 115.23 (2C), 70.04 (1C), 41.58 (1C), 34.20 (1C).

N-(4-(benzyloxy)phenethyl)-2-chloro-4-(cyclohexylamino)pyrimidine-5-carboxamide (7c)

To a stirring solution of (5b) (1.745 mmol) in 5 ml of anhydrous tetrahydrofuran was added cyclohexylamine (2.095 mmol), and the resulting suspension was stirred at ambient temperature for 24 h. The solution was filtered to remove the undissolved salts, and the filtrate was concentrated in vacuo to produce a crude yellow oil. Purification of the oil was performed using flash chromatography on silica gel (ethyl acetate/hexanes 0-30%) to afford 7c (1.260 mmol, 75%) as a white solid. ¹H NMR (400 MHz, CDCl₃) δ 8.78 (d, J=7.9 Hz, 1H), 7.99 (s, 1H), 7.44-7.27 (m, 4H), 6.99 (dd, J=78.1, 8.6 Hz, 4H), 6.40 (s, 1H), 5.01 (s, 2H), 4.13-4.00 (m, 1H), 3.58 (q, J=6.8 Hz, 2H), 2.82 (t, J=6.9 Hz, 2H), 2.00-1.90 (m, 2H), 1.72 (dd, J=13.3, 4.1 Hz, 2H), 1.61 (dd, J=12.3, 3.5 Hz, 1H), 1.48-1.18 (m, 5H). ¹³C NMR (101 MHz, CDCl₃) δ 166.06 (1C), 162.43 (1C), 160.84 (1C), 157.64 (1C), 154.63 (1C), 136.89 (1C), 130.64 (1C), 129.70 (2C), 128.55 (2C), 127.96 (1C), 127.46 (2C), 115.13 (2C), 106.81 (1C), 70.04 (1C 4H), 49.04 (1C), 40.99 (1C), 34.57 (s, 4H), 32.36 (2C), 25.53 (1C), 24.44 (2C).

N-(4-(benzyloxy)phenethyl)-2-chloro-4-(neopentylamino)pyrimidine-5-carboxamide (7d)

The product was obtained from 5b and neopentylamine (0.598 mmol) in a similar manner as described for the preparation and purification of 7c, affording the pure compound 7d (0.393 mmol, 79%). ¹H NMR (400 MHz, CDCl₃) δ 9.08 (t, J=5.6 Hz, 1H), 8.02 (s, 1H), 7.43-7.27 (m, 4H), 7.09 (d, J=8.6 Hz, 2H), 6.88 (d, J=8.6 Hz, 2H), 6.69 (t, J=5.6 Hz, 1H), 5.00 (s, 2H), 3.59 (q, J=6.8 Hz, 2H), 3.32 (d, J=5.8 Hz, 2H), 2.82 (t, J=7.0 Hz, 2H), 0.99 (s, 9H). ¹³C NMR (101 MHz, CDCl₃) δ 166.14 (1C), 162.28 (1C), 162.24 (1C), 157.59 (1C), 154.67 (1C), 136.90 (1C), 130.76 (s, 1H), 129.73 (2C), 128.56 (2C), 127.96 (1C), 127.48 (2C), 115.07 (2C), 106.86 (1C), 70.04 (1C), 52.07 (1C), 41.05 (1C), 34.56 (1C), 31.84 (1C), 27.41 (3C).

N-(4-(benzyloxy)phenethyl)-2-cyano-4-(cyclohexylamino)pyrimidine-5-carboxamide (8a)

A mixture of 7c (0.433 mmol), sodium cyanide (0.866 mmol), and 1,4-diazabicyclo[2.2.2]octane (DABCO) (0.866 mmol) in anhydrous dimethylsulfoxide (2 mL) was heated in a CEM microwave at 80° C. for 10 minutes. After cooling to ambient temperature, the solution was diluted with 8 mL of water and the precipitate filtered. The precipitate was dissolved in dichloromethane and purified by flash chromatography (ethyl acetate/hexanes 0-50%) to afford pure 8a (0.294 mmol, 68%) as a white powder. ¹H NMR (400 MHz, CDCl₃) δ 8.75 (d, J=7.4 Hz, 1H), 8.12 (s, 1H), 7.49-7.28 (m, 5H), 7.12 (d, J=8.4 Hz, 2H), 6.94 (d, J=8.4 Hz, 2H), 6.18 (s, 1H), 5.05 (s, 2H), 4.14-3.97 (m, 1H), 3.63 (q, J=6.5 Hz, 2H), 2.85 (t, J=6.7 Hz, 2H), 2.03-1.91 (m, 2H), 1.75 (dd, J=9.3, 4.0 Hz, 2H), 1.69-1.59 (m, 1H), 1.52-1.19 (m, 6H). ¹³C NMR (101 MHz, CDCl₃) δ 165.44 (1C), 159.49 (1C), 157.75 (1C), 152.78 (1C), 145.87 (1C), 136.83 (1C), 130.29 (1C), 129.66 (2C), 128.56 (2C), 127.98 (1C), 127.44 (2C), 115.77 (1C), 115.25 (2C), 109.63 (1C), 70.04 (1C), 60.37 (1C), 49.35 (1C), 41.02 (1C), 34.41 (1C), 32.27 (1C), 29.67 (2C), 25.47 (1C), 24.47 (1C).

N-(4-(benzyloxy)phenethyl)-2-cyano-4-(neopentylamino)pyrimidine-5-carboxamide (8b)

The product was obtained from 7d (0.376 mmol) in a similar manner as described for the preparation and purification of 8a, affording the pure compound 8b (0.293 mmol, 78%). ¹H NMR (400 MHz, CDCl₃) δ 9.03 (t, J=5.5 Hz, 1H), 8.13 (s, 1H), 7.44-7.27 (m, 5H), 7.11 (d, J=8.4 Hz, 2H), 6.91 (d, J=8.5 Hz, 2H), 6.45 (t, J=5.6 Hz, 1H), 5.01 (s, 2H), 3.63 (q, J=6.6 Hz, 2H), 3.33 (d, J=6.0 Hz, 2H), 2.85 (t, J=6.9 Hz, 2H), 0.98 (s, 9H). ¹³C NMR (101 MHz, CDCl₃) δ 165.55 (1C), 160.93 (1C), 157.66 (1C), 152.88 (1C), 145.66 (1C), 136.87 (1C), 130.51 (1C), 129.73 (2C), 128.57 (2C), 127.98 (1C), 127.45 (2C), 115.79 (1C), 115.17 (2C), 109.72 (1C), 70.04 (1C), 52.01 (1C), 41.14 (1C), 34.44 (1C), 31.97 (1C), 27.35 (3C).

2-cyano-4-(cyclohexylamino)-N-(4-hydroxyphenethyl)pyrimidine-5-carboxamide (9a)

To a solution of 8a (0.439 mmol) in dry dichloromethane (3 mL) under N₂ atmosphere was added boron tribromide (1 M in dichloromethane, 0.439 mmol) dropwise at room temperature. The reaction mixture was monitored by TLC; upon completion (approximately 2 hours), ice-water was added, and the mixture was extracted three times with ethyl acetate. The combined organic layers were washed with water (20 mL), brine (20 mL), dried over anhydrous Na₂SO₄, and evaporated under reduced pressure to afford a crude yellow oil. The oil was purified by flash chromatography (methanol/dichloromethane 0-5%) to afford compound 9a (0.068 mmol, 16%). ¹H NMR (400 MHz, CD₃OD) δ 8.36 (s, 1H), 7.03 (d, J=8.4 Hz, 2H), 6.70 (d, J=8.5 Hz, 2H), 4.05-3.96 (m, J=13.2, 9.6, 3.7 Hz, 1H), 3.54-3.44 (m, J=13.4, 5.7 Hz, 2H), 2.76 (t, J=7.3 Hz, 2H), 1.98-1.90 (m, J=12.3, 3.3 Hz, 2H), 1.81-1.69 (m, J=9.1, 4.2 Hz, 2H), 1.68-1.56 (m, J=8.6, 4.1 Hz, 1H), 1.51-1.22 (m, 6H). ¹³C NMR (101 MHz, CD₃OD) δ 165.74 (1C), 159.36 (1C), 155.57 (1C), 153.33 (1C), 145.05 (1C), 129.23 (2C), 114.83 (2C), 110.29 (1C), 49.06 (1C), 41.17 (1C), 34.15 (1C), 31.79 (2C), 25.20 (1C), 24.14 (2C). HRMS (ESI) calc. for C₂₀H₂₃N₅O₂ (M +H)⁺ 366.1925, found 366.1926.

2-cyano-N-(4-hydroxyphenethyl)-4-(neopentylamino)pyrimidine-5-carboxamide (9b)

The product was obtained from 8b (0.371 mmol) in a similar manner as described for the preparation and purification of 9a, affording the pure compound 9b (0.043 mmol, 38%). ¹H NMR (400 MHz, CDCl₃) δ 8.99 (t, J=5.4 Hz, 1H), 8.09 (s, 1H), 7.04 (d, J=8.4 Hz, 2H), 6.75 (d, J=8.4 Hz, 2H), 6.31 (t, J=5.4 Hz, 1H), 5.64 (s, 1H), 3.68-3.56 (m, 2H), 3.33 (d, J=6.0 Hz, 2H), 2.82 (t, J=6.7 Hz, 2H), 0.97 (s, 9H). ¹³C NMR (101 MHz, CDCl₃) δ 165.46 (1C), 160.90 (1C), 154.68 (1C), 152.64 (1C), 130.07 (1C), 129.89 (2C), 115.72 (2C), 115.57 (1C), 109.66 (1C), 52.02 (1C), 41.10 (1C), 34.33 (1C), 31.97 (1C), 27.32 (3C). HRMS (ESI) calc. for C₁₉H₂₃N₅O₂ (M+H)⁺ 354.1925, found 354.1921.

### Radiochemical Syntheses

Syntheses of 2-cyano-4-(cyclohexylamino)-N-(4-[¹¹C]methoxyphenethyl)-pyrimidine-5-carboxamide ([¹¹C]1) and 2-cyano-N-(4-[¹¹C]methoxyphenethyl)-4-(neopentylamino)pyrimidine-5-carboxamide ([¹¹C]2) (FIG. 1)

All steps were performed using the integrated functions of the TRACERLab FX C Pro synthesis system. [¹¹C]CO2 from the cyclotron (GE PETtrace 6) was converted by gas-phase methods to [¹¹C]methyl iodide. The reaction of gaseous [¹¹C]CH₃I with 2-cyano-4-(cyclohexylamino)-N-(4-hydroxyphenethyl)pyrimidine-5-carboxamide (9a) or 2-cyano-N-(4-hydroxyphenethyl)-4-(neopentylamino)pyrimidine-5-carboxamide (9b) took place in the FX C Pro reaction vessel. The vessel was charged with 1.0 mg of precursor (9a or 9b), 10 μL of 7.5 M NaOH, and 100 μL of dimethylformamide. [¹¹C]CH₃I was delivered from the methyl iodide synthesis unit and bubbled into the reaction vessel at a fixed flow rate of 15 mL/min for 3 min; then the flow was stopped, and the solution was stirred for an additional 2 min at ambient temperatures. The reaction mixture was diluted with 1 mL of 50/50 acetonitrile/water and injected into a preparatory HPLC column (Phenomenex Luna C18(2), 10×150 mm, eluted with 60% acetonitrile, 40% H₂Om 20 mM NH₄OAc at 6 mL/min). The fractions corresponding to [¹¹C]1 or [¹¹C]2 (typically eluting out between 9.5 and 11.5 min) were collected and transferred into a dilution flask containing sterile water (50 mL). The resulting solution was transferred through a Waters C18 1 cc Sep-Pak to collect the desired product. The C18 Sep-Pak was then washed with sterile water (10 mL) to remove unwanted hydrophilic impurities and residual acetonitrile to waste. [¹¹C]1 or [¹¹C]2 were eluted off into a collection vial with ethanol for injection, USP (0.5 mL) and 0.9% sodium chloride for injection, USP (4.5 mL). The final formulation (5 mL) was then passed into a sterile vial to provide [¹¹C]1 (2.47±0.84 GBq, n=15) or [¹¹C]2 (2.26±0.81 GBq, n=5). Radiochemical purities (>99%) and specific activities ([¹¹C]1; 560±180 GBq/μmol: [¹¹C]2; 488 ±129 GBq/μmol) were determined using analytical HPLC (45% acetonitrile: 25% H₂O, 20 mM NH₄OAc, oven 30° C., 254 nm, flow rate: 1.0 mL/min, RT=5-6 min, RCP≧99.7%. Based on approx. 3 Ci of [¹¹C]CO₂, overall radiochemical yields were 2-3% (end-of-synthesis) and were not optimized.

### Biology

### Rat Biodistribution Studies

The whole body biodistribution of radioactivity after intravenous injection of [¹¹C]1 was determined in rats. Animals (N =6, female CD rats) were lightly anesthetized (isoflurane) and injected via the tail vein with 1.85-4.44 MBq of radioligand formulated in 5% ethanol/saline. At selected times (5 and 40 min), groups (N=3) of animals were anesthetized, euthanized (decapitation) and samples of tissues removed. Tissue samples were weighed, counted for carbon-11, and the data calculated as % injected dose/g.

### MicroPET Imaging Studies

Studies were done using imaging with Concorde Microsystems R4 and P4 PET scanners. Rats were anesthetized with isoflurane and maintained on 1% isoflurane throughout the imaging period. The animals were placed on their dorsal side in the scanner, and body temperature was maintained using a heating pad. A catheter was inserted into the tail vein for administration of radioactive solutions. Following a measured transmission scan, solutions of radioligands ([¹¹C]1 or ([¹¹C]2, 37-185 MBq in 0.5-1 mL volumes) were injected via the catheter, followed by a 1 ml flush of saline. For the blocking study, a 2 mg/kg dose of unlabeled compound 2 was administered 10 minutes prior to the injection of the radiolabeled [¹¹C]2. Emission data was collected for 60 minutes (12×5 min fames). The emission data was corrected for decay, dead time and random coincidences, then reconstructed using iterative ordered subset expectation maximization-maximum a posteriori (OSEM-MAP) yielding a reconstructed image resolution of approximately 1.4 mm. Volumetric regions of interest (ROIs) were defined using hand-drawn circular ROIs placed on the summed images, and the time-activity curves determined for each ROI (ASI Pro VM software: Siemens Medical Systems, Malvern, Pa.).

### Example III

The family of cysteine proteases known as the cathepsins (11 members: cathepsins B, C, F, H, K, L, O, S, V, W and X) have widespread functions in the mammalian body and are involved in both normal and pathological biochemical processes. Only recently have the cathepsins become of interest as potential targets for development of disease-specific in vivo imaging agents (see, e.g., Wadas, et al., J Nucl Med. 2009; 50:1873-1880; Edem, et al., Nucl Med Biol. 2010; 37:681; Ren, et al., PLoS ONE. 2011; e28029; Loser, et al., Chem Med Comm. 2013; 8:1330-1344; Ogbomo, et al., Nucl Med Biol. 2013; 40:606-617).The clear involvement of cathepsin K in osteolytic bone diseases, and the potential importance of new cathepsin K inhibitors as treatment for osteoporosis, prompted the present experiments into the potential of radiolabeling cathepsin K inhibitors.

The target compounds (1 and 2; see FIG. 1) were selected from a large number of potential cathepsin K inhibitors which have now been described in the literature (see, e.g., Altmann, et al., J Med Chem. 2007; 50:591-594; WO 2004/020441; Robichaud, et al., Bioorg Med Chem Lett. 2007; 17:3146-3151; Teno, et al., Bioorg Med Chem Lett. 2007; 17:6096-6100; Teno, et al., Bioorg Med Chem Lett. 2008; 18:5833-5836). The selection was based on synthetic accessibility to the required precursors for radiolabeling, high apparent affinities for human cathepsin K (in vitro IC₅₀ values of 0.022 nM and 0.003 nM for 1 and 2, respectively), and 10- to 100-fold selectivities over cathepsins L and S (see, e.g., Altmann, et al., J Med Chem. 2007; 50:591-594).

The syntheses of the authentic ligands 1 and 2, and the required precursors for carbon-11 labeling 9a and 9b, were done following slight modifications of the published methods (see Scheme 1). The syntheses began with commercially available 2,4-dichloropyrimidine-5-carbonyl chloride (3) which underwent amide bond formation with 2-(4-methoxyphenyl)ethylamine (4a) or 2-(4-(benzyloxy)phenyl)ethanamine hydrochloride (4b), in the presence of diiopropylethyl amine, to afford compounds 5a and 5b. Subsequent reaction of 5a or 5b with cyclohexylamine or neopentyl amine providing compounds 7a-d in good yields. The 2-substituted pyrimidines (7a-d) were then converted into the corresponding cyanopyrimidines by displacement of the 4-chloro substituent with cyanide. The reaction was performed using microwave heating techniques in the presence of DABCO (1,4-diazabicyclooctane) and NaCN and yielded cyanides 1, 2, 8a, and 8b. As a final step in preparation for carbon-11 labeling, the nitriles 8a-b underwent debenzylation in the presence of 1 M boron tribromide in dichloromethane to yield the necessary desmethyl precursors 9a and 9b. Careful chromatographic purifications provided the desmethyl compounds in low yield.

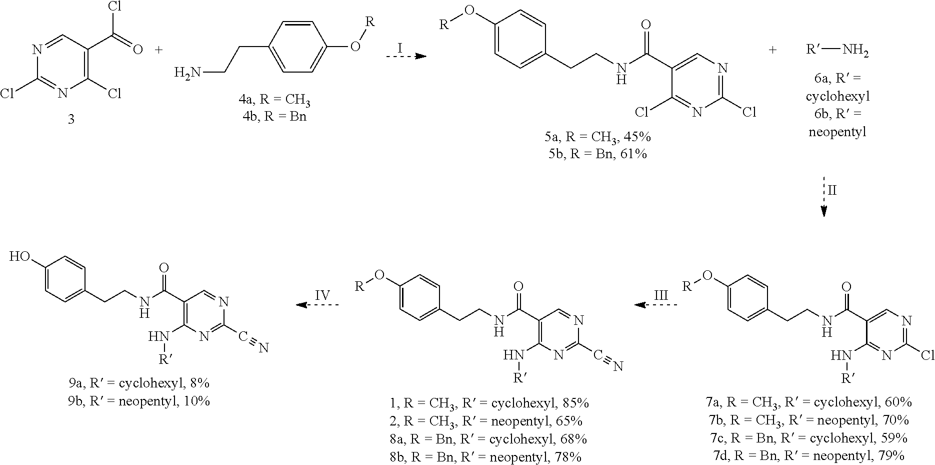

The radiochemical syntheses of [¹¹C]1 and [¹¹C]2 were done using the automated Tracerlab FXcPro system and standard reaction conditions used for alkylation reactions with [¹¹C]methyl iodide. Emphasis was placed on preparation of radiochemically pure (>99%) and high specific activity products, and synthesis yields were low and not optimized.

The in vivo biological properties of these potential radioligands was first evaluated by determining the whole-body biodistribution of [¹¹C]1 in rats (see Table 1). No unusual early uptake (5 min) or significant retention (40 min) was observed for any specific organ in the rat body. Highest concentrations were observed in abdominal organs (e.g., liver) and very low levels in muscle and bone (a large bone sample of the femur in its entirety).

TABLE 1
Tissue biodistribution of radioligand [¹¹C]1 in female rats
following intravenous injection.

| | |
|---|---|
| 5 min | 40 min |

| | avg | s.d. | avg | s.d. |
|---|---|---|---|---|
| Brain | 0.43 | 0.05 | 0.11 | 0.0015 |
| Heart | 1.23 | 0.18 | 0.19 | 0.02 |
| Lung | 0.68 | 0.09 | 0.16 | 0.019 |
| Liver | 3.02 | 0.21 | 0.68 | 0.033 |
| Pancreas | 1.68 | 0.19 | 0.56 | 0.069 |
| Spleen | 1.03 | 0.079 | 0.22 | 0.015 |
| Adrenal | 3.57 | 0.86 | 0.51 | 0.053 |
| Kidney | 2.03 | 0.17 | 0.41 | 0.042 |
| Muscle | 0.11 | 0.008 | 0.11 | 0.024 |
| Bone | 0.26 | 0.018 | 0.14 | 0.028 |
| Blood | 0.24 | 0.007 | 0.11 | 0.015 |

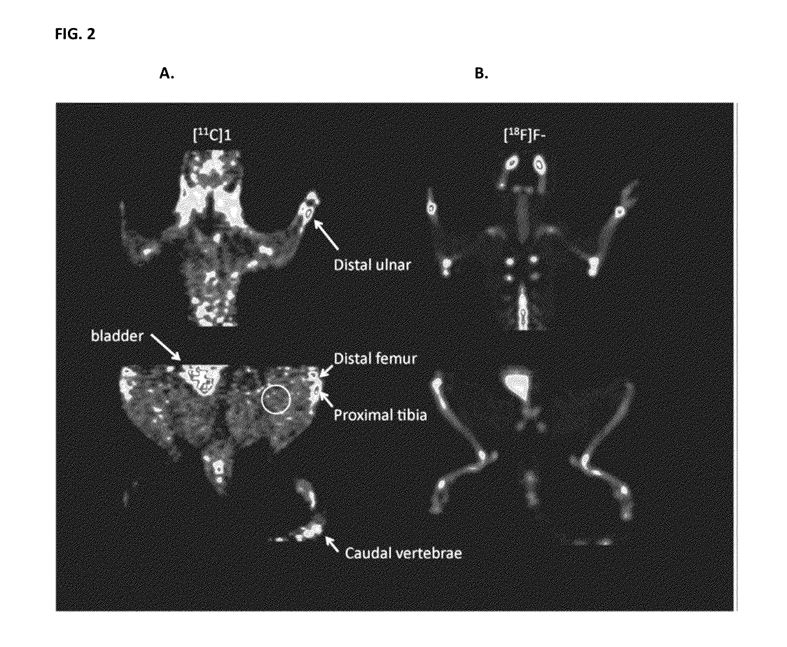

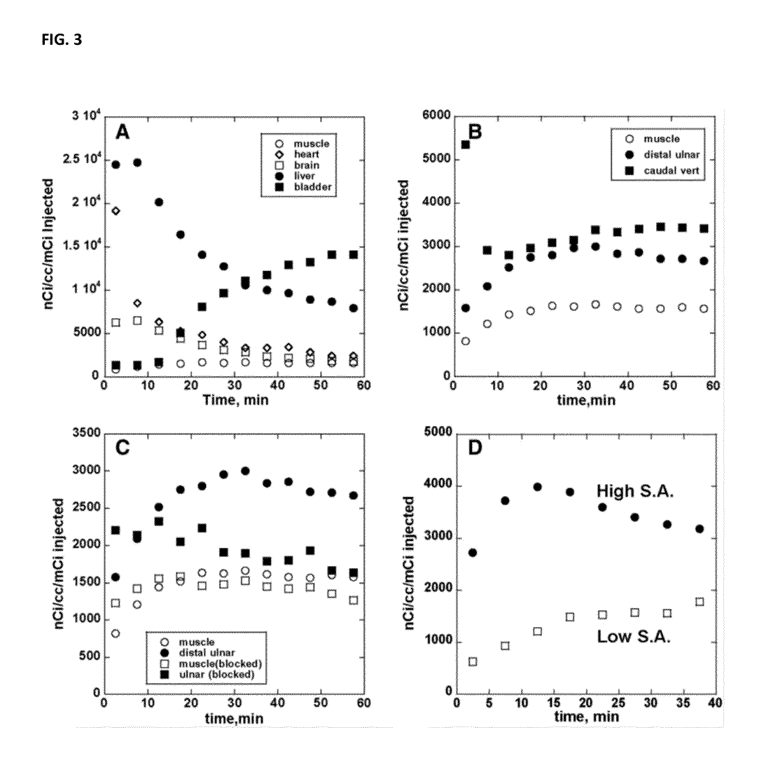

The potential for imaging the osteoclasts in bone was then evaluated in normal rats using microPET imaging; representative images of localization of [¹¹C]1 in the rat are shown in FIG. 2 (equivalent results were obtained for [¹¹C]2). As an aid in identification of appropriate skeletal features, a bone scan using [¹⁸F]fluoride ion was completed in several animals following the [¹¹C]radiotracer study. Consistent with the biodistribution data, high early uptake was observed in the liver and low in muscle, with steadily increasing concentrations in the bladder (FIG. 3A). The uptake and clearance from the heart and brain are consistent with a moderately lipophilic radiotracer. For both radioligands, localization of radioactivity in actively growing bone regions (e.g., distal ulnar, carpal, distal and proximal humeral, distal femur, proximal tibia, tail vertebrae) was clearly evident, reaching levels 2 to 3 times higher than in non-target regions such as muscle (FIG. 3B). The lack of clearance of radioactivity from the bone regions is consistent with the irreversible nature of binding of these cathepsin K inhibitors.

That the bone uptake of [¹¹C]1 and [¹¹C]2 represented saturable, high-affinity binding was demonstrated in two different ways. The administration of a blocking dose of unlabeled ligand 2, ten minutes prior to the injection of [¹¹C]2, resulted in a reduction of bone uptake of radioactivity to essentially levels equivalent to background in non-target tissues (FIG. 3C). Uptake and retention of radioactivity in the non-target areas were unaltered by dosing with unlabeled drug. In a slightly different experiment, studies were done using high (414 GBq/μmol) and low (10.8 GBq/μmol) specific activity preparations of radioligand [¹¹C]1. The administration of the low specific activity dose, representing co-injection of 14 micrograms/kg of unlabeled 1, produced a similar nearly full block of the uptake and retention of the radioligand into the target bone regions (FIG. 3D). Thus, for both [¹¹C]1 and [¹¹C]2, the localization of radioactivity into specific bone regions appears to represent specific binding that can be readily competed for by pre- or co-injection of unlabeled compound.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical sciences are intended to be within the scope of the following claims.

We claim:

1. A composition comprising a radiolabeled compound selected from the group consisting of: 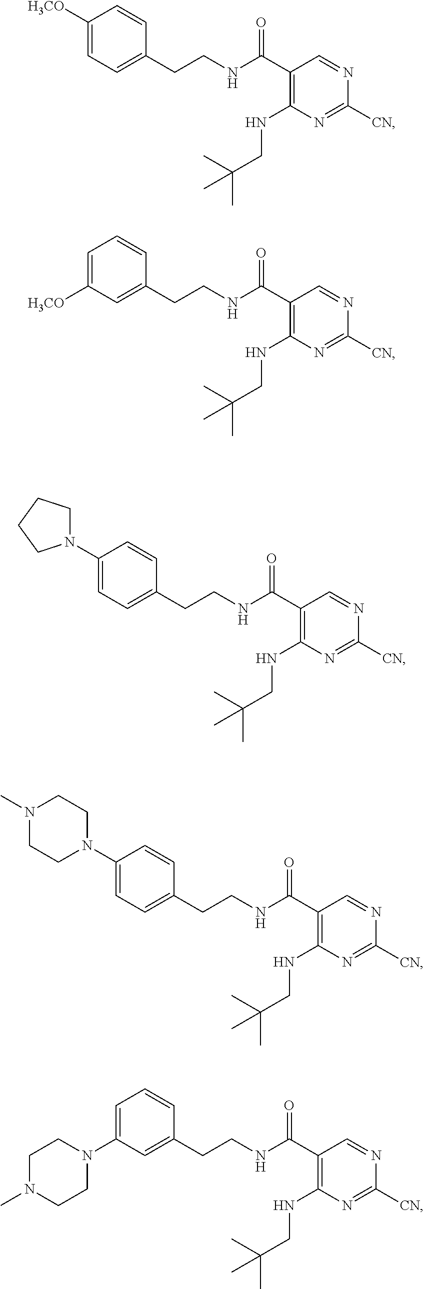 [SMILES: COc1ccc(CCNC(=O)c2cnc(C#N)nc2NCC(C)(C)C)cc1 ; COc1cccc(CCNC(=O)c2cnc(C#N)nc2NCC(C)(C)C)c1 ; CC(C)(C)CNc1nc(C#N)ncc1C(=O)NCCc1ccc(N2CCCC2)cc1 ; CN1CCN(c2ccc(CCNC(=O)c3cnc(C#N)nc3NCC(C)(C)C)cc2)CC1 ; CN1CCN(c2cccc(CCNC(=O)c3cnc(C#N)nc3NCC(C)(C)C)c2)CC1] 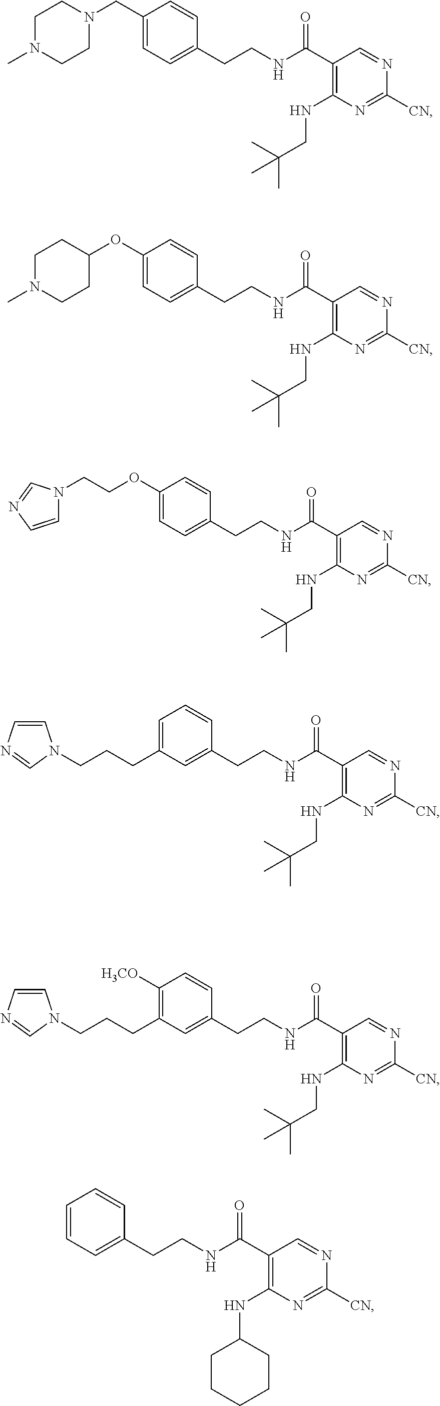 [SMILES: CN1CCN(Cc2ccc(CCNC(=O)c3cnc(C#N)nc3NCC(C)(C)C)cc2)CC1 ; CN1CCC(Oc2ccc(CCNC(=O)c3cnc(C#N)nc3NCC(C)(C)C)cc2)CC1 ; CC(C)(C)CNc1nc(C#N)ncc1C(=O)NCCc1ccc(OCCn2ccnc2)cc1 ; CC(C)(C)CNc1nc(C#N)ncc1C(=O)NCCc1cccc(CCCn2ccnc2)c1 ; COc1ccc(CCNC(=O)c2cnc(C#N)nc2NCC(C)(C)C)cc1CCCn1ccnc1 ; N#Cc1ncc(C(=O)NCCc2ccccc2)c(NC2CCCCC2)n1] 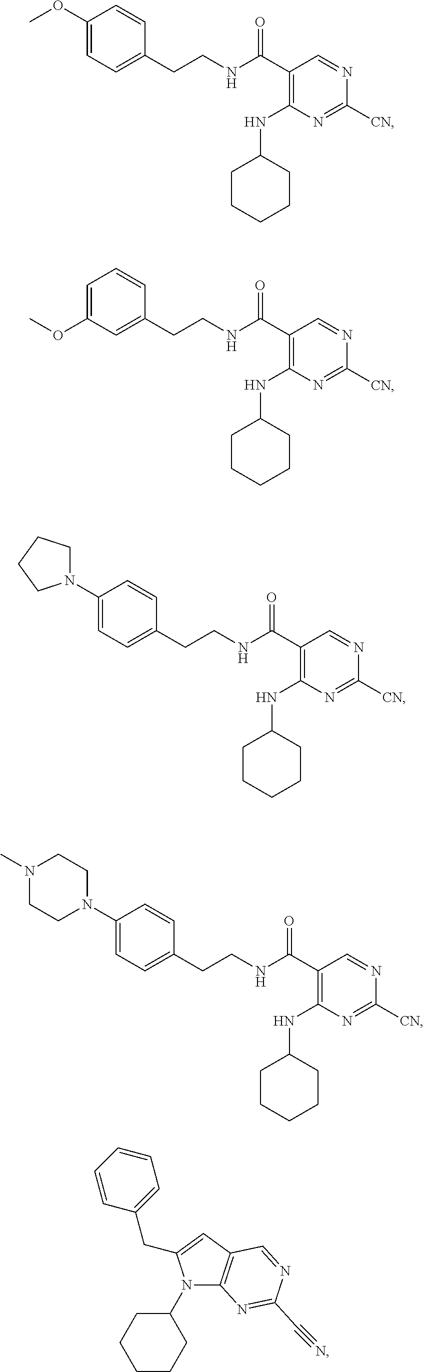 [SMILES: COc1ccc(CCNC(=O)c2cnc(C#N)nc2NC2CCCCC2)cc1 ; COc1cccc(CCNC(=O)c2cnc(C#N)nc2NC2CCCCC2)c1 ; N#Cc1ncc(C(=O)NCCc2ccc(N3CCCC3)cc2)c(NC2CCCCC2)n1 ; CN1CCN(c2ccc(CCNC(=O)c3cnc(C#N)nc3NC3CCCCC3)cc2)CC1 ; N#Cc1ncc2cc(Cc3ccccc3)n(C3CCCCC3)c2n1] 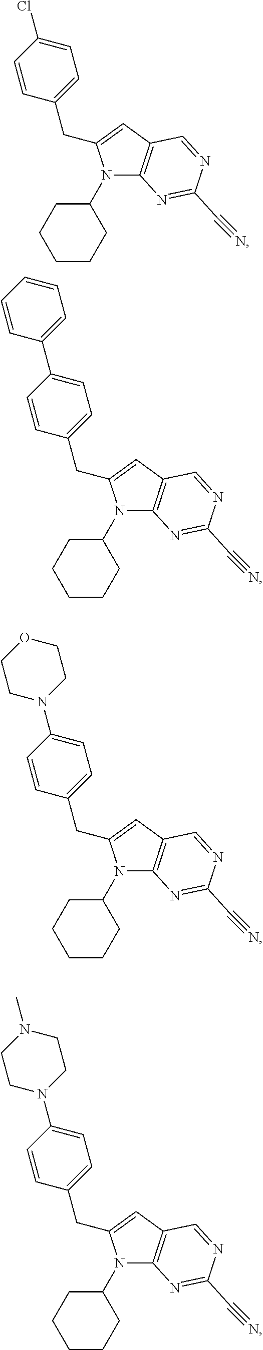 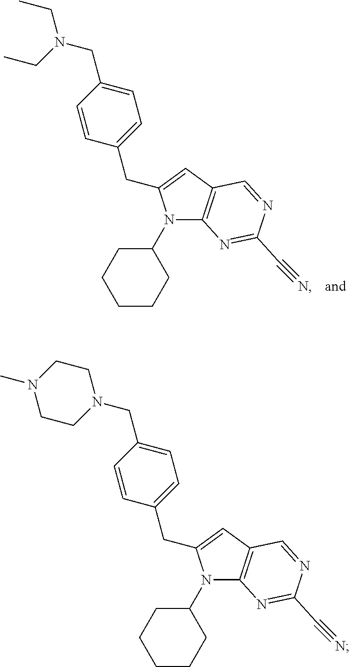 [SMILES: CCN(CC)Cc1ccc(Cc2cc3cnc(C#N)nc3n2C2CCCCC2)cc1 ; CN1CCN(Cc2ccc(Cc3cc4cnc(C#N)nc4n3C3CCCCC3)cc2)CC1]
wherein the radiolabeled compound is radiolabeled with either ¹¹C, ¹⁸F or ¹²³I;
wherein the radiolabeled compound is a cathepsin K inhibitor.

2. The composition of claim 1, wherein the radiolabeled compound is selected from the group consisting of: 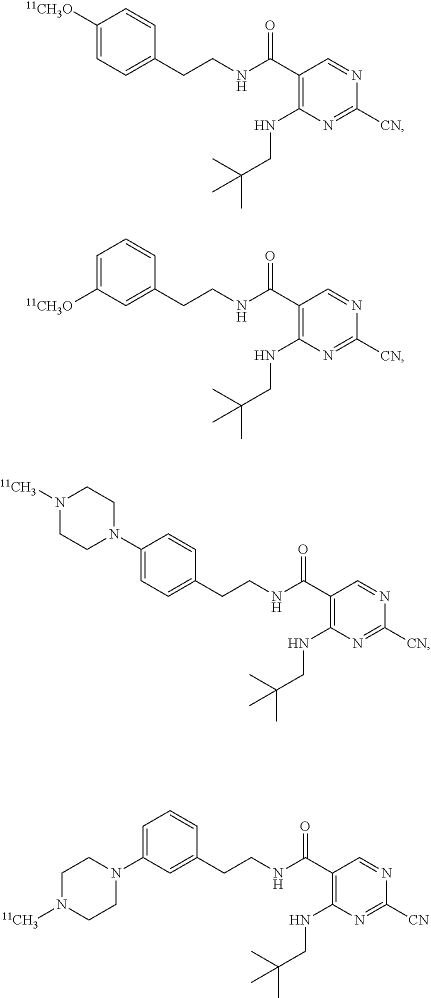 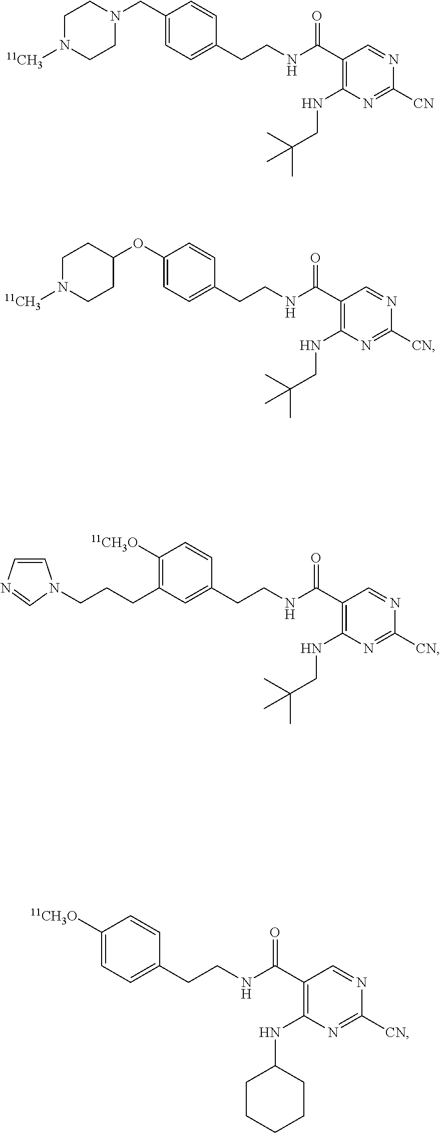 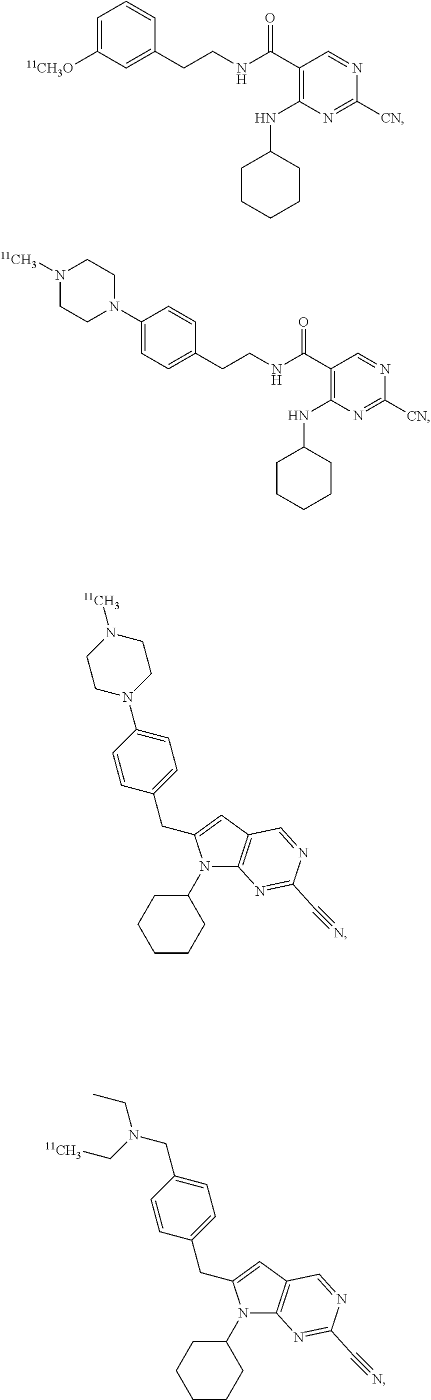 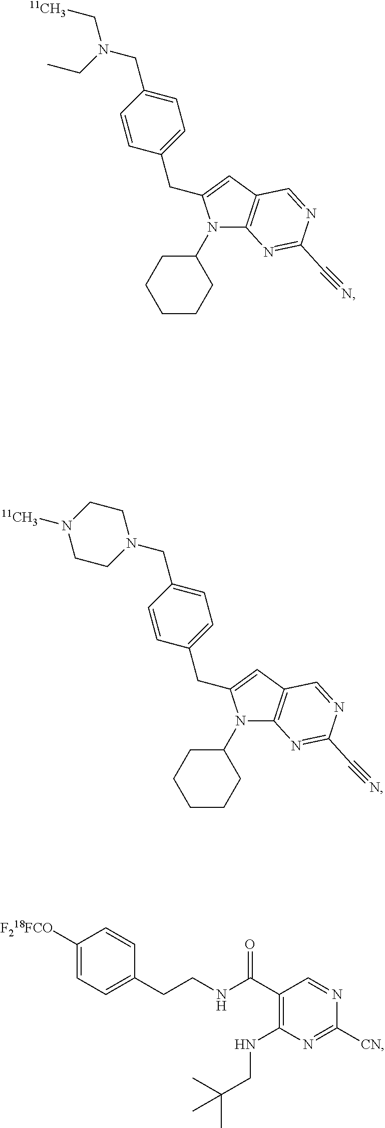 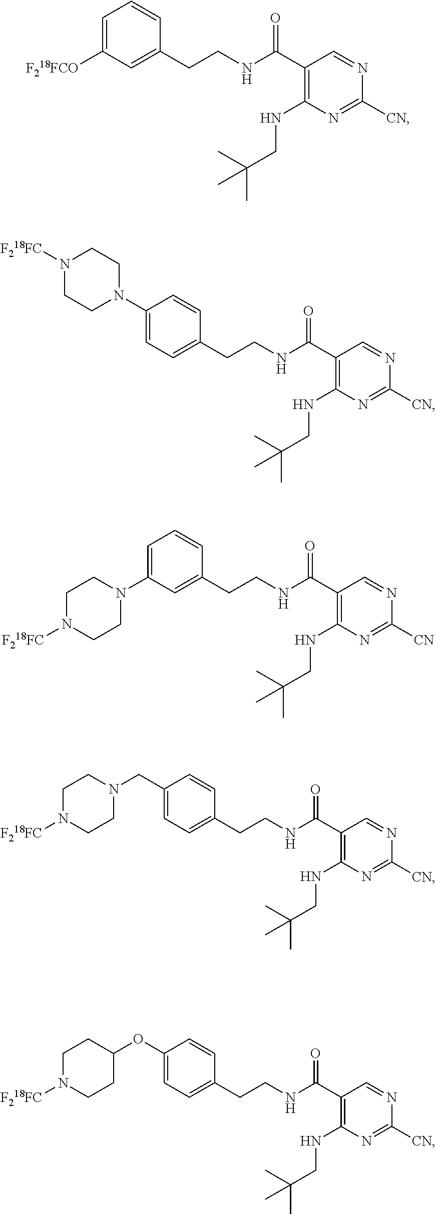 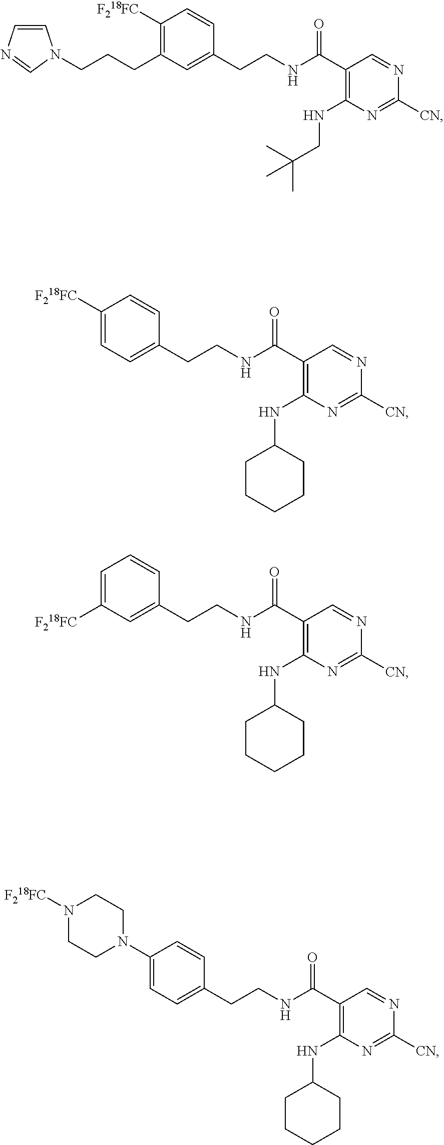 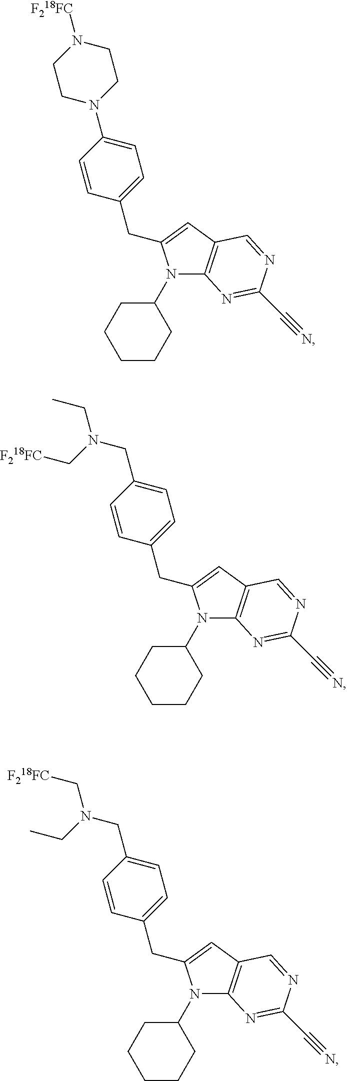 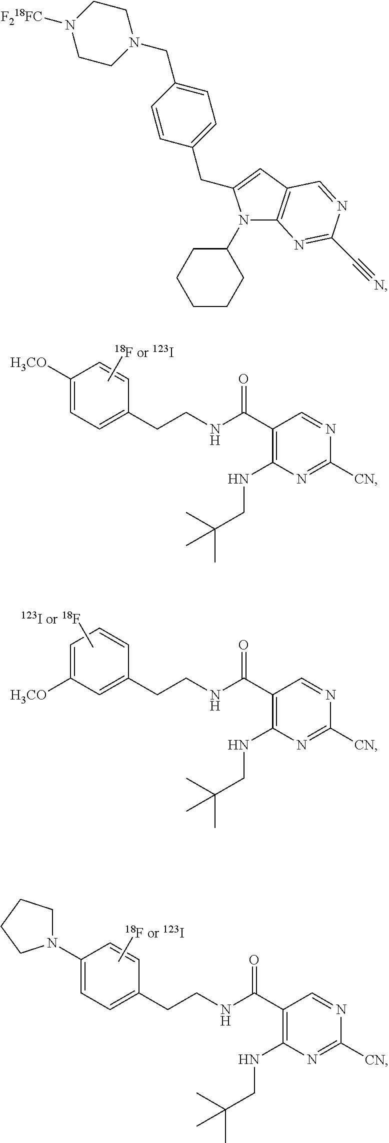 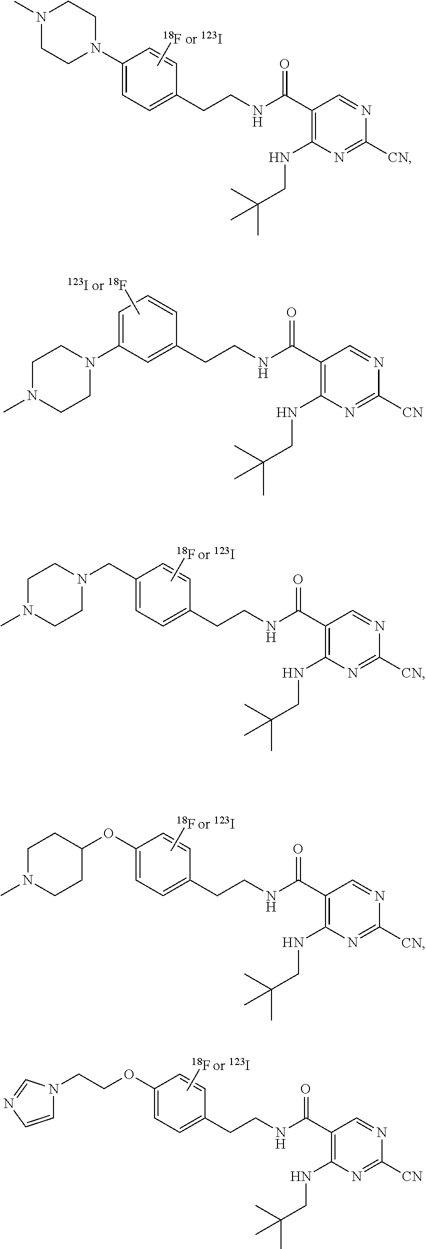 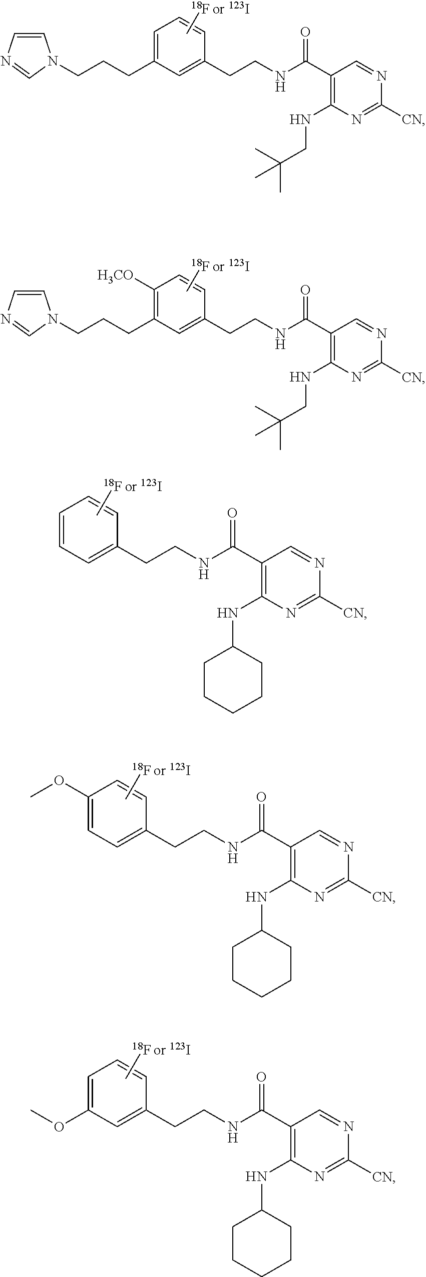 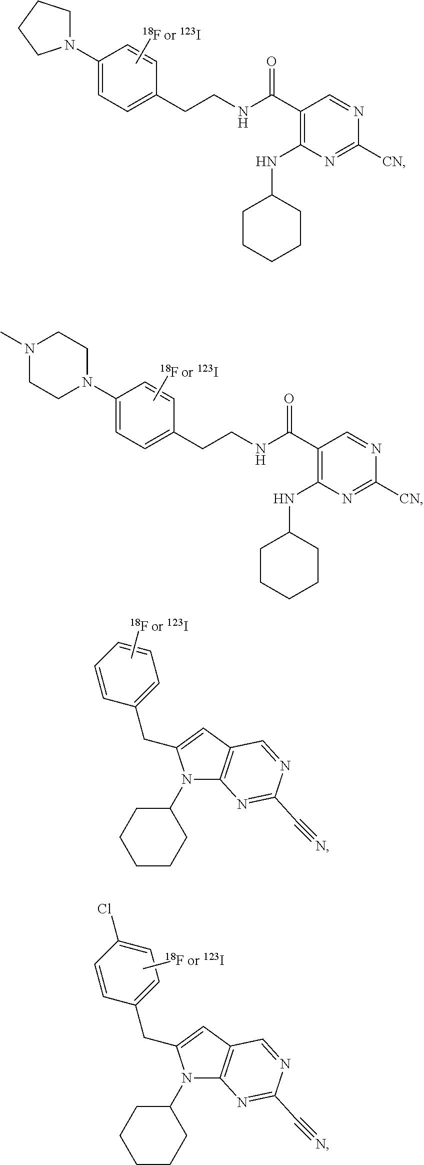 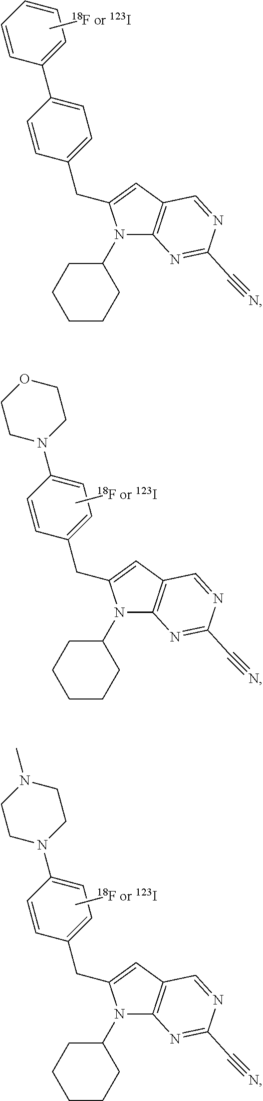 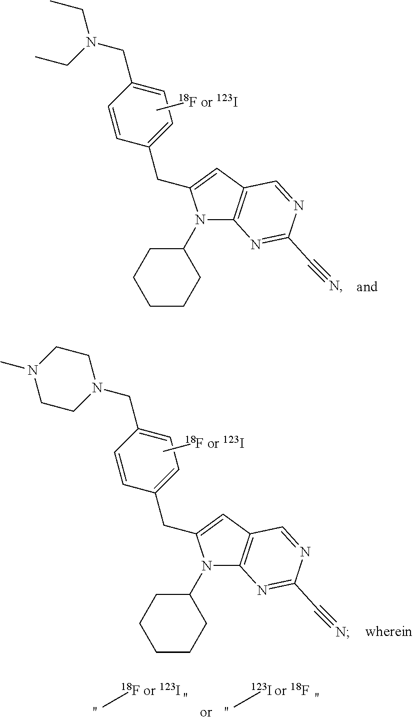
indicates that a Hydrogen molecule on the respective aromatic ring is replaced with either ¹⁸F or ¹²³I.

3. The composition of claim 1, wherein the composition is an imaging composition.

4. A method of imaging comprising:
(a) administering a composition comprising a radiolabeled compound of claim 1 to a subject; and
(b) detecting gamma radiation emitted by said composition and forming an image therefrom.

5. The method of claim 4, wherein said subject is a human being.

6. The method of claim 4, wherein said imaging is PET imaging or SPECT imaging.

7. A method of assessing osteoclast activity in a subject, comprising
(a) administering a composition comprising a radiolabeled compound of claim 1 to a subject;
(b) detecting gamma radiation emitted by said composition and forming an image therefrom;
(c) quantifying the amount of cathepsin K activity in said subject based upon said detecting and forming of an image, and
(d) assessing osteoclast activity in said subject based upon said quantifying of the amount of cathepsin K activity in said subject.

8. The method of claim 7, further comprising:
(e) comparing said quantified cathepsin K activity with one or more established cathepsin K activity norms associated with osteoporosis, and
(f) determining the presence or absence of osteoporosis in said subject based upon said comparing.

9. The method of claim 7, wherein said imaging is PET imaging or SPECT imaging.

10. The method of claim 8, wherein said one or more established cathepsin K activity norms associated with osteoporosis are one or more selected from the group consisting of established cathepsin K activity norms for subjects having a pre-osteoporosis condition, established cathepsin K activity norms for subjects diagnosed with moderate osteoporosis, established cathepsin K activity norms for subjects diagnosed with severe osteoporosis, established cathepsin K activity norms for subjects having a low risk for osteoporosis onset, established cathepsin K activity norms for subjects having a moderate risk for osteoporosis onset, and established cathepsin K activity norms for subjects having a high risk for osteoporosis onset.

11. The method of claim 10, wherein said subject is a human being.
